(19) United States
(12) Patent Application Publication
(10) Pub. No.: US 2025/0161051 A1
(43) Pub. Date: May 22, 2025

(54) IMPLANT TENSIONING

(71) Applicant: Edwards Lifesciences Innovation (Israel) Ltd., Caesarea (IL)

(72) Inventors: Or Cohen, Modi'in (IL); Haim Brauon, Beit Dagan (IL); Assaf Sharon, Tel Aviv (IL); Tal Sheps, Givat Shmuel (IL); Eyal Shaolian, Newport Beach, CA (US); Nicholas Scott Steenwyk, Minneapolis, MN (US); Bezalel Haberman-Browns, Pardes Hanna (IL)

(21) Appl. No.: 19/023,131

(22) Filed: Jan. 15, 2025

Related U.S. Application Data
(63) Continuation of application No. PCT/IB2023/057093, filed on Jul. 11, 2023.
(60) Provisional application No. 63/389,609, filed on Jul. 15, 2022.

(51) Int. Cl. A61F 2/24; A61B 17/00; A61B 17/04

(52) U.S. Cl. CPC A61F 2/2466; A61B 17/0469; A61B 2017/00398; A61B 2017/0496; A61F 2220/0016; A61F 2220/0075; A61F 2250/0012

(57) ABSTRACT

Tensioning devices are described that include a handle, a line carriage configured to be coupled to a tensioning line, and an electrical motor assembly configured to axially translate the tensioning line relative to the handle to thereby modify a tension in the tensioning line. Other implementations are also described, including a tensioning device including a handle, a line carriage configured to be coupled to a tensioning line, and a manually-manipulatable actuator configured to be selectively transitioned between a fine-tensioning configuration and a gross-tensioning configuration.

### RELATED APPLICATION(S)

This application is a continuation of International Patent Application No. PCT/IB2023/057093, filed Jul. 11, 2023, and entitled IMPLANT TENSIONING, which claims priority to U.S. Provisional Patent Application Ser. No. 63/389,609, filed on Jul. 15, 2022, and entitled IMPLANT TENSIONING, the complete disclosures of each of which are hereby incorporated by reference in their entirety.

### BACKGROUND

Annuloplasty generally involves the remodeling of tissue of a native heart valve annulus. Annulus remodeling can be performed by pulling tissue around/of the annulus to a new shape. Tensioning wires/lines connecting tissue anchors and/or other implant devices can be used to facilitate medical procedures, such as annuloplasty or other remodeling procedures.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure can be included in the examples summarized here.

Described herein are one or more methods and/or devices to facilitate the tensioning of tension wires/lines associated with implant devices/assemblies, such as annuloplasty implants. Implementations of the present disclosure further relate to quick-release mechanisms for selectively implementing gross- and fine-tuning of tension in a tension wire/line, and associated handle devices.

In some implementations, the present disclosure relates to a tensioning device comprising a handle, a line carriage configured to be coupled to a tensioning line, and a manually-manipulatable actuator configured to be selectively transitioned between a fine-tensioning configuration and a gross-tensioning configuration.

In some implementations, the actuator comprises an internal channel configured to have a bolt member associated with the line carriage disposed therein, the internal channel including threading configured to mesh with threading associated with the bolt member. For example, in some examples, in the fine-tensioning configuration, threading of the bolt is meshed with the threading associated with the actuator, and in the gross-tensioning configuration, the threading of the bolt is not meshed with the threading associated with the actuator.

In some implementations, the actuator can be configured to transition between the fine-tensioning configuration and the gross-tensioning configuration by tilting relative to an axis of the tensioning device. For example, the internal channel can comprise two merged pseudo channels that are angled relative to one another.

In some implementations, the two merged pseudo channels include a first pseudo channel that is parallel to an axis of the actuator and a second pseudo channel that is angled relative to the axis of the actuator.

In some implementations, the threading of the actuator can be associated with only a first pseudo channel of the two merged pseudo channels. For example, in some implementations, in a first axial portion of the first pseudo channel, the threading is associated only with a first diametrical half of the first pseudo channel, and in a second axial portion of the first pseudo channel, the threading is associated only with a second diametrical half of the first pseudo channel that is opposite the first diametrical half.

In some implementations, the actuator comprises a first axial segment configured to rotate about an axis of the actuator relative to a second axial segment of the actuator. For example, each of the first axial segment and the second axial segment can comprise internal threading associated with only one diametrical half thereof. In some implementations, in the fine-tensioning configuration, the internal threading of the first axial segment does not circumferentially overlap with the internal threading of the second axial segment, and in the gross-tensioning configuration, the internal threading of the first axial segment circumferentially overlaps with the internal threading of the second axial segment.

In some implementations, in the gross-tensioning configuration, at least one of the bolt member or the actuator can be biased such that the bolt member contacts non-threaded internal walls of the first and second axial segments, such that the bolt member can axially slide within the actuator.

In some implementations, the tensioning line can comprise one or more filaments having variable electrical resistance. For example, an electrical resistance of the one or more filaments may change based on an amount of stretch in the tensioning line.

In some implementations, the present disclosure relates to a tensioning device comprising a handle, a line carriage configured to be coupled to a tensioning line, and an electrical motor assembly configured to axially translate the tensioning line relative to the handle to thereby modify a tension in the tensioning line.

In some implementations, the electrical motor assembly can comprise a motor gear meshed with a gear rotationally fixed to an internally-threaded rotational-to-linear translation structure. For example, the electrical motor assembly can further comprise an externally-threaded axial translating rod, the rod being coupled to the line carriage. In some implementations, the tensioning line is configured to be disposed through an inner channel of the translating rod and the line carriage.

In some implementations, the tensioning device further comprises an electrical tension-measuring means configured to generate signals indicating the tension in the tensioning line.

In some implementations, the tensioning device can further comprise a user output means configured to provide user output indicating a tension condition of the tensioning line.

In some implementations, the tension-measuring means comprises one or more contacts configured to provide signals indicating an electrical resistance of the electrical motor assembly.

In some implementations, the tension-measuring means comprises a strain gauge. For example, the strain gauge can be physically coupled between the tensioning line and the line carriage.

In some implementations, the tensioning line can comprise one or more filaments having an electrical resistance that varies based on an amount of stretch in the tensioning line. For example, the one or more filaments can be braided with sutures or wires.

In some implementations, the tensioning line further comprises one or more signal filaments. For example, the one or more signal filaments can be fiber optic filaments.

For purposes of summarizing the disclosure, certain aspects, advantages, and novel features have been described. It is to be understood that not necessarily all such advantages may be achieved in accordance with any particular implementation. Thus, the disclosed implementations may be carried out in a manner that achieves or optimizes one advantage or group of advantages as taught herein without necessarily achieving other advantages as may be taught or suggested herein.

### BRIEF DESCRIPTION OF THE DRAWINGS

Various implementations are depicted in the accompanying drawings for illustrative purposes and should in no way be interpreted as limiting the scope of the inventions. In addition, various features of different disclosed implementations can be combined to form additional implementations, which are part of this disclosure. Throughout the drawings, reference numbers may be reused to indicate correspondence between reference elements.

FIG. 1 shows cardiac anatomy and certain implant devices and instrumentation for contracting an annulus of a native heart valve in accordance with one or more implementations.

FIG. 2 shows an annuloplasty implant implanted in a native heart valve annulus in accordance with one or more implementations.

FIGS. 3A and 3B show a tissue anchor implant assembly in non-cinched and cinched configurations, respectively, in accordance with one or more implementations.

FIGS. 4A and 4B show portions of a tissue anchor implant assembly and cinching system in accordance with one or more implementations.

FIG. 5 shows an implant tensioning system in accordance with one or more implementations.

FIGS. 6A and 6B show stages of an implant tensioning process in accordance with one or more implementations.

FIGS. 7A, 7B, and 7C are schematic illustrations of a tensioning device in accordance with one or more implementations.

FIGS. 8A, 8B, and 8C show perspective, side cross-sectional, and axial views of a tensioning actuator in accordance with one or more implementations.

FIG. 9A shows a portion of a tensioning device in accordance with one or more implementations.

FIGS. 9B and 9C show perspective and axial views, respectively, of a tensioning actuator in an engaged configuration in accordance with one or more implementations.

FIGS. 9D and 9E show perspective and axial views, respectively, of a tensioning actuator in a disengaged configuration in accordance with one or more implementations.

FIG. 9F is a diagram showing an axial view of engaged and disengaged tension wire carriage channels for the tensioning actuator of FIGS. 9A-9E in accordance with one or more implementations.

FIGS. 10A, 10B, 10C, and 10D provide a flow diagram illustrating a process for tensioning a tension wire associated with an implant device in accordance with one or more implementations.

FIGS. 11A, 11B, 11C, and 11D provide images of certain devices/components corresponding to operations of the process of FIGS. 10A, 10B, 10C, and 10D in accordance with one or more implementations.

FIGS. 12A and 12B show cutaway and exploded views, respectively, of components of a motorized tensioning device in accordance with one or more implementations.

FIGS. 13A and 13B show components of a motorized tensioning assembly/device in various actuation states in accordance with one or more implementations.

FIG. 14 shows a tensioning device with motor-resistance-based tension reading functionality in accordance with one or more implementations.

FIG. 15 shows a tensioning device with strain-gauge-based tension reading functionality in accordance with one or more implementations.

FIG. 16 shows a tensioning line including strain indication features in accordance with one or more implementations.

FIG. 17 shows a tensioning line including strain indication features in accordance with one or more implementations.

FIG. 18 shows an implant device including a cable-type tensioning line in accordance with one or more implementations.

FIGS. 19A, 19B, and 19C show cross-sectional views of tensioning lines in accordance with one or more implementations.

FIG. 20 shows a cross-sectional view of a tensioning line in accordance with one or more implementations.

FIGS. 21A, 21B, and 21C show cross-sectional views of strands including special filaments in accordance with one or more implementations.

FIG. 22 shows a braided line in accordance with one or more implementations.

FIG. 23 shows a cutaway view of a filament of a tensioning line in accordance with one or more implementations.

### DETAILED DESCRIPTION

The headings provided herein are for convenience only and do not necessarily affect the scope or meaning of the claimed invention.

Although certain implementations and examples are disclosed below, inventive subject matter extends beyond the specifically disclosed implementations to other alternative implementations and/or uses and to modifications and equivalents thereof. Thus, the scope of the claims that may arise herefrom is not limited by any of the particular implementations described below. For example, in any method or process disclosed herein, the acts or operations of the method or process can be performed in any suitable sequence and are not necessarily limited to any particular disclosed sequence. Various operations may be described as multiple discrete operations in turn, in a manner that may be helpful in understanding certain implementations; however, the order of description should not be construed to imply that these operations are order dependent.

The techniques, methods, operations, steps, etc. described or suggested herein or in the references incorporated herein can be performed on a living subject (e.g., human, other animal, etc.) or on a simulation, such as a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

Additionally, the structures, systems, and/or devices described herein can be embodied as integrated components or as separate components. For purposes of comparing various implementations, certain aspects and advantages of these implementations are described. Not necessarily all such aspects or advantages are achieved by any particular implementation. Thus, for example, various implementations can be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that can be similar in one or more respects. However, with respect to any of the implementations disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Certain standard anatomical terms of location are used herein to refer to the anatomy of animals, and namely humans, with respect to the preferred implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The present disclosure relates to tensioning devices, systems, and methods for adjusting tension in a wire or other line, which can be coupled to one or more tissue anchors or other implant device(s)/component(s). Such implementations can involve linear/axial actuation of the tensioned line to modify tension in the line. The term “line” is used herein according to its broad and ordinary meaning and may refer to any elongate wire, tether, cord, strip, strand, suture, rope, filament, tie, string, ribbon, strap, or portion thereof, or other type/form of material used in medical procedures to tension, tether, cinch, secure, align, tie, hold, or otherwise control/manipulate implant devices or components (e.g., tissue anchors). Furthermore, implementations of the present disclosure can be implemented in connection with non-surgical and/or non-biological line/wire tensioning. Furthermore, in some contexts herein, the terms “tether,” “wire,” and “line” can be used substantially interchangeably. In addition, use of the singular form of any of the line-related terms listed above, including the terms “tether” and “wire,” may be used to refer to a single line/cord, or to a portion thereof.

In some aspects, the present disclosure relates to systems, devices, and methods for adjusting the tension of a line associated with an implant device/assembly. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

The above-referenced method(s) can be performed on a living animal or on a simulation, such as on a cadaver, cadaver heart, anthropomorphic ghost, simulator (e.g., with body parts, heart, tissue, etc. being simulated).

Certain implementations are disclosed herein in the context of cardiac implant devices. However, although certain principles disclosed herein are particularly applicable to the anatomy of the heart, it should be understood that tissue-anchor-based implant devices in accordance with the present disclosure may be implanted in, or configured for implantation in, any suitable or desirable anatomy.

### Valvular Heart Disease and Annulus Reshaping

The anatomy of the heart is described below to assist in the understanding of certain inventive concepts disclosed herein. In humans and other vertebrate animals, the heart generally comprises a muscular organ having four pumping chambers, wherein the blood flow therein is at least partially controlled by various heart valves, namely, the aortic, mitral (or bicuspid), tricuspid, and pulmonary valves. The valves may be configured to open and close in response to pressure gradients present during various stages of the cardiac cycle (e.g., relaxation and contraction) to control the flow of blood to respective regions of the heart and/or to blood vessels (e.g., pulmonary, aorta, etc.). The contraction of the various heart muscles may be prompted by signals generated by the electrical system of the heart, which is discussed in detail below.

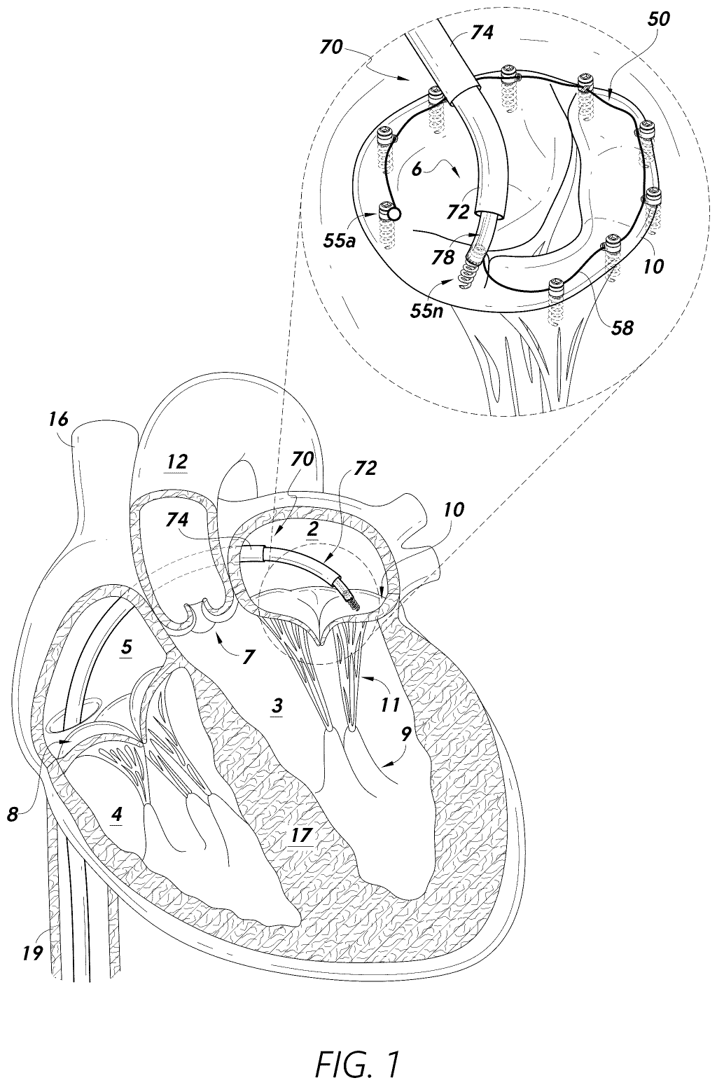

FIG. 1 illustrates an example representation of a heart 1 and associated anatomy having various features relevant to certain implementations of the present inventive disclosure. Generally, the heart 1 includes four chambers, namely the left atrium 2, the left ventricle 3, the right ventricle 4, and the right atrium 5. In terms of blood flow, blood generally flows from the right ventricle 4 into the pulmonary artery via the pulmonary valve (not shown for clarity), which separates the right ventricle 4 from the pulmonary artery and is configured to open during systole so that blood may be pumped toward the lungs and close during diastole to prevent blood from leaking back into the heart from the pulmonary artery. The pulmonary artery carries deoxygenated blood from the right side of the heart to the lungs.

In addition to the pulmonary valve, the heart 1 further includes the tricuspid valve 8, the aortic valve 7, and the mitral valve 6. The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally have associated therewith a relatively dense fibrous/collagenous ring-type structure, referred to herein as the valve annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage.

With respect to heart valves and associated interventions (e.g., annuloplasty procedures), particular reference is made herein to the mitral valve 6. However, it should be understood that any description herein of anatomy and/or devices or procedures associated with the mitral valve can apply to the other valves of the heart (e.g., tricuspid, aortic); references to the mitral valve specifically are for conveniences and/or due to particular relevance thereof.

With reference to the mitral valve 6, the annulus 10 thereof attaches the mitral leaflets 13 and the left atrium 2 to the ostium of the left ventricle 3 and the aortic root. Under normal conditions, the mitral valve 6 undergoes significant dynamic changes in shape and size throughout the cardiac cycle. These changes are primarily due to the dynamic motion of the surrounding mitral valve annulus 10. Throughout the cardiac cycle, the annulus 10 generally undergoes a sphincter motion, narrowing down the orifice area during systole to facilitate coaptation of the leaflets 13 and widening during diastole to allow for relatively easy diastolic filling of the left ventricle 3. This motion can be further enhanced by a pronounced three-dimensional configuration during systole, which may embody a characteristic saddle shape. The shape and form of the annulus 10 throughout the cardiac cycle can affect proper leaflet coaptation and/or tissue stresses. Disfunction of a heart valve and/or associated leaflets (e.g., pulmonary valve disfunction) can result in valve leakage and/or other health complications.

The atrioventricular (i.e., mitral and tricuspid) heart valves generally are coupled to a collection of chordae tendineae 11 and papillary muscles 9 for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle 17, referred to as the septum, separates the left 2 and right 5 atria and the left 3 and right 4 ventricles.

Various problems can disrupt blood flow through the valves of the heart. For example, regurgitation, which is also referred to as valve insufficiency or incompetence, occurs when a valve does not close properly and allows blood to leak backward instead of moving in the proper one-way flow. Regurgitation can cause a decrease in the amount of blood that ultimately travels to the body's organs. In order to compensate for regurgitation, the heart may work harder, which in time can cause enlargement/dilation of the heart and reduced cardiac output. In some cases, ischemic heart disease can cause valvular regurgitation. For example, mitral regurgitation can be caused by the combination of ischemic dysfunction of the papillary muscles and left ventricular dilation that can present in ischemic heart disease, with the subsequent displacement of the papillary muscles and the dilatation of the mitral valve annulus. Valve problems can be present at birth or caused by infections, heart attacks, or heart disease or damage.

Certain heart valve diseases/dysfunction can be treated through the implementation of certain annuloplasty treatments, which can help a deformed valve annulus to regain the physiological form and function of a normal, healthy valve (e.g., mitral valve, tricuspid valve, etc.) apparatus. For example, annuloplasty treatments can involve the implantation of a prosthetic annuloplasty device (e.g., ring-shaped device, “annuloplasty ring”, annuloplasty device, etc.). Annuloplasty treatments can serve to restore/remodel the annular dimensions of a heart valve annulus, which can promote proper leaflet coaptation and/or provide a broader surface of coaptation. Annuloplasty procedures can be used to effectively remodel the annulus to restore the size and physiologic shape (e.g., a D- or kidney-type shape) of the annulus and/or decreases the overall circumference of the annulus.

In some cases, an annuloplasty ring/device can be constructed from a core material, such as silicone or metal, that provides the desired rigidity; annuloplasty rings can be designed to be relatively flexible, semirigid, or rigid. Some annuloplasty rings are sheathed in a cloth material (e.g., Dacron or polyester), through which anchoring sutures may be placed. Annuloplasty rings can be formed in various sizes, rigidities, and shapes, and can comprise a partial band or complete ring. The size of an annuloplasty ring can be selected/determined based at least in part on the particular dimensions of the target heart valve/annulus (e.g., height of the anterior leaflet, intercommissural distance, intertrigonal distance, etc.).

Annuloplasty ring implantation procedures can be implemented by accessing the heart valve through the chest wall. For example, open-heart surgical access can be utilized to access the heart and the target valve. However, minimally invasive techniques wherein one or more relatively smaller cuts in the chest can be used to access the valve may reduce trauma.

Implementations of the present disclosure can be used in transcatheter annuloplasty interventions, wherein an implant device/assembly is advanced to the target valve annulus through the vasculature of the patient, which may be accessed through the femoral vein or other access site/path. Transcatheter annuloplasty procedures can be preferable to surgical and minimally-invasive procedures due to associated decreased risks and trauma to the patient; transcatheter annuloplasty procedures in accordance with aspects of the present disclosure can be particularly desirable for the treatment of patients with valvular regurgitation deemed inoperable or at high surgical risk.

The less invasive, percutaneous (e.g., transcatheter) implantation of annuloplasty devices in accordance with implementations of the present disclosure provides for relatively safer treatment when compared with open-heart surgery. Furthermore, such procedures may allow for relatively earlier indication for treatment to improve the chance of prognostic benefit. Transcatheter annuloplasty devices comprising tissue anchors coupled by tensioning tether/line connectors in accordance with implementations of the present disclosure, may produce results similar to those of surgical annuloplasty rings, such as with respect to the ability to decrease the septolateral dimension and increase leaflet coaptation. An example system that may be utilized in connection with the tensioning solutions presented herein is the Cardioband Mitral Reconstruction System by Edwards Lifesciences, which is a percutaneous surgical-like direct annuloplasty device that can be implanted in a beating heart. Such devices may be implanted on the annulus of a native valve under fluoroscopic and/or transesophageal echocardiographic (TEE) guidance. Following the implantation of tissue anchors associated with the implant device in or near the native valve annulus, the device can be contracted using wire/line tensioning to remodel the annulus and improve mitral regurgitation.

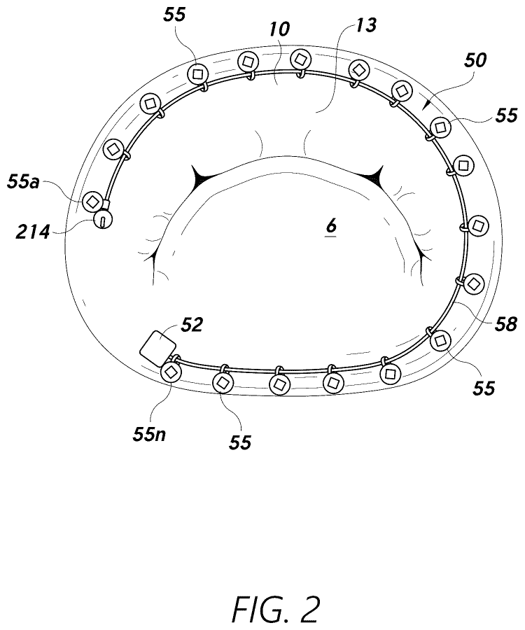

With further reference to FIG. 1, an example annuloplasty implant device 50 is shown, which provides an adjustable annuloplasty system that can be adjusted, after implantation of a plurality of tissue anchors 55, by adjusting the tension of a wire or other type of line 58 that is coupled to each of the anchors 55. FIG. 2 shows an overhead view of the annuloplasty implant device 50 implanted in a native heart valve annulus 10 in accordance with one or more implementations. In the image of FIG. 2, the tensioning wire 58 has been tensioned and locked, and the delivery system 70 has been withdrawn.

In some implementations, the anchors 55 can be secured to the native annulus 10 and/or other anatomy surrounding the valve orifice/leaflets 13, such that tensioning the line 58 draws the anchors 55 closer to one another and/or inward towards the axis/center of the valve 6. In some implementations, the implant 50 can include a sleeve (e.g., polyester) around at least a portion of the line 58 and/or anchors 55. Tensioning of the line 58 can effectively shorten the implant 50 to thereby adjust the implant size to the patient's needs. Tensioning can be performed under imaging (e.g., echo) guidance as a means of producing and confirming the desired regurgitation reduction.

In some implementations, the implant 50 can be delivered to the target anatomy using a delivery system 70, which can comprise, for example, one or more delivery and/or guide catheters 72 and/or an access sheath 74, any of which can be steerable. For mitral repair, at least a portion of the delivery system 70 can be advanced through the interatrial septum.

In some implementations, the catheter(s) 72 can include a steerable guide catheter, as well as a flexible tube configured to be advanced through the guide catheter in order to facilitate delivery and implantation of the implant 50 therefrom. During the delivery, at least a portion of a possibly steerable distal end of such flexible tube can be deployed from the distal end of the guide catheter for advancement to the annulus 10 of the target valve (e.g., mitral valve 6).

In some implementations, the implant 50 can comprise a plurality of implantable tissue anchors 55, which can comprise metal or other biocompatible material. In some implementations, the anchors 55 are corkscrew-type tissue anchors having a proximal drive head, which can be engaged by a driver tool 78 to rotate and/or embed the anchors into the target tissue.

In some implementations, the delivery system 70 can include anchor delivery shaft(s) (e.g., pusher(s)) for advancing the anchors from a distal end of the delivery system 70. In some implementations, the tissue anchors 55 comprise stainless steel anchors that are between 5-10 mm in length (e.g., approximately 6 mm). The anchors 55 can be used to fasten the connecting line/wire 58 to the target tissue (e.g., annulus 10).

Although 18 anchors 55 are shown in FIG. 2, it should be understood that any number of tissue anchors can be implemented, such as any number between 8-20. The anchors 55 can be repositionable and/or retrievable prior to full deployment of the implant 50 as shown in FIG. 2.

Certain processes/operations can be implemented to deliver and anchor the implant device 50 in a subject (e.g., in a living subject, in a simulation, etc.). For example, the delivery system 70 can be advanced toward the annulus 10 through any suitable access path, such as through one or more veins and/or arteries of the patient. In some implementations, an outer catheter/sheath 74 can be advanced through the patient's vasculature into the right atrium 5 and through the interatrial septum until its distal end is positioned in the left atrium 2. A steerable distal end portion of the outer catheter/sheath can then be steered such that it is positioned in a desired spatial orientation within the left atrium 2. The steering procedure can be performed with the aid of imaging, such as fluoroscopy, transesophageal echo, and/or echocardiography.

Access to the right atrium 5 can be made using any suitable or desirable access path, such as through the femoral vein and/or through arterial access. It should be understood that any suitable point of origin can be utilized. For example, access can be made by introduction into the femoral vein of the patient, through the inferior vena cava 19, into the right atrium 5, and into the left atrium 2 transseptally (e.g., through the fossa ovalis). In some implementations, access can be made through the basilic vein, the subclavian vein, the superior vena cava 16, and into the right atrium 5. In some implementations, access can be made via the jugular vein, the subclavian vein, the superior vena cava 16, and into the right atrium 5 and/or the left atrium 2.

In some implementations, a guidewire can be advanced to the target position within the left atrium 2 through the selected access path. In some implementations, a relatively stiff guidewire can be utilized for the purpose of traversing the interatrial septum.

In some implementations, the delivery system 70 can be advanced over the guidewire into the left atrium 2. In some implementations, the delivery catheter 72 can be placed over the anterior commissure to provide a suitable attack position for deployment of the first anchor 55a.

Verification of the first anchoring location can be obtained using imaging.

In some implementations, the first anchor 55a is placed close to the leaflet hinge, as anterior as possible in the annulus, close to the anterior commissure.

In some implementations, the implant 55 can include a terminal stopper (e.g., crimp ball) 214 that is distal to the most-distal anchor 55a and prevents the line 58 from being pulled through the anchor/line coupling means (e.g., eyelet, hook, or the like) associated with the anchor 55a.

In some implementations, the anchors can be delivered sequentially. In some implementations, multiple anchors can be delivered simultaneously.

In some implementations, the first anchor 55a can be delivered and implanted using the anchor delivery drive 78, which can be provided inside the catheter(s) 72. The anchors 55 can be released after proper anchoring is confirmed with push-and-pull testing under imaging guidance (e.g., echocardiography, fluoroscopy).

In some implementations, after proper anchoring of one anchor, the catheter(s) 72 can be navigated/advanced (e.g., using steering knobs of a proximal handle/system, or the like) to the next anchoring point along the annulus 10. Such actions can be repeated until the implant catheter reaches the last anchoring site, where the last anchor 55n is deployed. The implant 50 can then be disconnected from the delivery catheter(s) 72 with the tensioning wire 58 passing from the implant 50 through the sheath 70 and the patient's anatomy to a location outside the body, wherein an adjustment tool can be advanced over the tensioning wire 58 to allow for adjustment in the tension of the wire/line 58.

In some implementations, the tension adjustment tool/device can be actuated/controlled to cause the tensioning wire/line 58 to be contracted. For example, rotation of a knob or other actuator associated with a handle of the tension adjustment tool/device can be executed to cause the tensioning of the wire/line 58.

After and/or during tensioning, adequate reduction of heart valve regurgitation or other defect can be assessed using imaging (e.g., echo) under beating heart conditions. When the tensioning has caused the appropriate implant size/diameter to be reached, the tensioning device/tool and/or other instrumentation can be used to cut and/or lock the tension in the wire/line 58, after which the non-implant instrumentation can be withdrawn from the patient, leaving the implant 50 disposed in/at the target anatomy with the desired degree of contraction.

In some implementations, the anchors 55 can be anchored sequentially around all or part (e.g., 40-90%, 50-70%, etc.) of the annulus 10, which is followed by the tensioning of the wire/line 58 in order to contract the annulus 10, as shown in FIG. 2. In some implementations, tensioning the wire 58 will be sufficient to adequately reduce or eliminate regurgitation through the native valve, in which case, the method/procedure may be concluded. In some implementations, if there is still a significant/undesirable amount of regurgitation at the native valve (e.g., shortly after the initial procedure steps/tensioning or after the passage of more time, such as days, weeks, months, or years later), a replacement prosthetic valve can be deployed in the native annulus.

In some implementations, the implant device 51 can advantageously increase leaflet coaptation and/or provide support to the posterior annulus against dilation. Compared to certain surgical annuloplasty ring devices, the annuloplasty implant 50 shown in FIG. 2, comprising a plurality of tissue anchors 55 coupled to a common tensioned wire/line 58, may advantageously provide improved flexibility to maintain the three-dimensional contour of the native annulus and some of its natural dynamics.

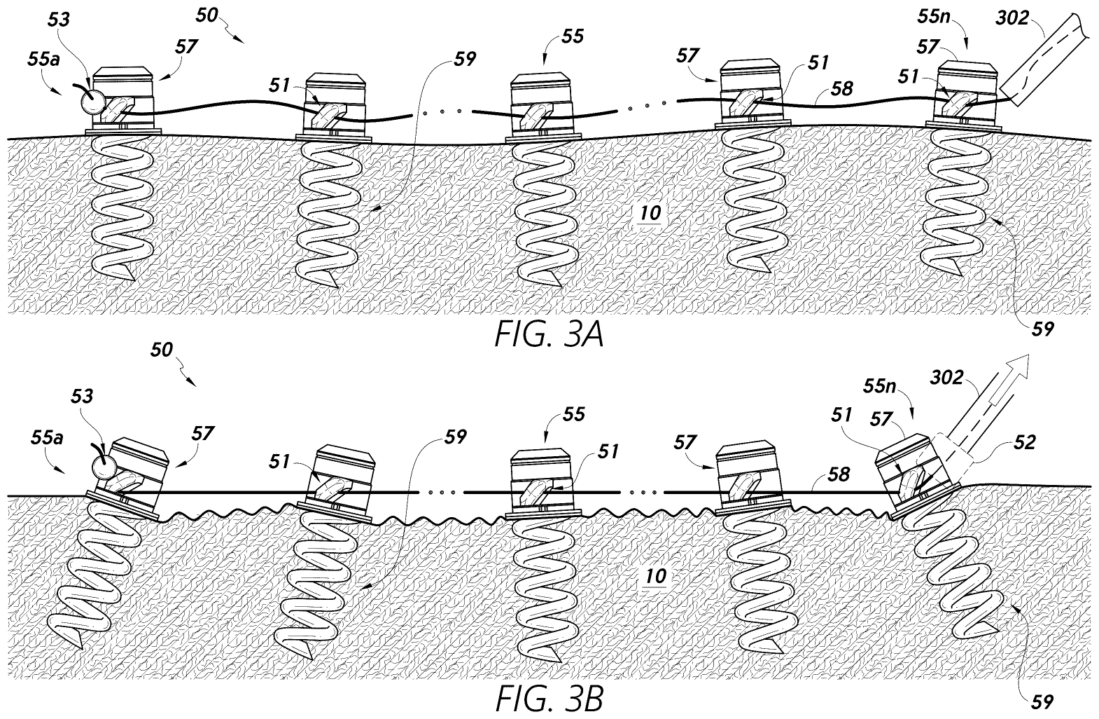

FIGS. 3A and 3B show an example tissue anchor implant assembly 50 in non-cinched and cinched configurations, respectively, in accordance with one or more implementations. That is, such figures show the anchors 55 of the implant device 50 embedded/anchored in the target tissue 10 (e.g., native annulus), wherein FIG. 3A shows the implant 50 after embedding of the anchors 55 but prior to tensioning of the wire/line 58. FIG. 3B shows the implant 50 after tensioning of the wire/line 58 in accordance with any of the tensioning solutions presented herein has been implemented to draw the anchors 55 together and reduce the size of the implant 50, and thereby reshape the annulus 10.

In some implementations, the anchors 55 can be embedded in the tissue 10 by, for example, rotating a drive head 57 of the anchors 55 such that a tissue-engaging element/portion 59 of the anchor 55 becomes driven into the tissue 10.

In some implementations, the tissue-engaging element/portion 59 of the anchors 55 can have a corkscrew form, as illustrated, in some implementations. Though the tissue-engaging portion can take and/or comprise other forms as well, e.g., a dart, staple, hook, needle, barb, arm, etc.

In some implementations, the wire/line 58 can be drawn proximally through eyelets or other line engagement/coupling features 51 of the respective anchors to secure the anchors to the wire/line 58. In some implementations, the line engagement/coupling feature 51 can be attached to the anchor 55 in a manner as to be rotatable around an axis of the anchor, which can provide independence between the rotational position of the feature 51 and that of the tissue-engaging portion 59 and/or drive head 57 of the anchor 55.

In FIG. 3A, multiple anchors 55 have been anchored to the tissue 10. After a desired number of anchors 55 have been anchored, an adjustment tool 302 can be introduced, which can be over and along a proximal portion of the wire/line 58, wherein such tool can be used to facilitate tensioning of the wire.

In some implementations, a reference force is provided (e.g., against a last anchor 55n to be anchored) by the tool 302 and/or component(s) thereof, while the wire 58 is pulled proximally. In some implementations, a first/distal end of the wire/line 58 cannot slide out of the first anchor 55a (e.g., through the line engagement/coupling feature 51) due to application of a fixation means/mechanism 53 (e.g., stopper) to prevent the wire/line 58 from being drawn through the coupling feature 51. For example, the fixation means/mechanism 53 can comprise stopper ball/form, as shown. Therefore, the tensioning of the wire/line 58 draws the anchors 55 relatively closer together, thereby contracting the tissue to which the anchors are anchored (see FIG. 3B).

In some implementations, the coupling features/means 51 of the anchors 55 can provide/allow for smooth sliding of the wire/line 58 through the coupling features/apertures 51 while the wire/line 58 is orthogonal to the anchors 55. In some implementations, the tension in the wire/line 58 can be locked using a locking clamp/mechanism 52, which can be coupled to the wire/line 58 proximal to the last anchor 55n. The excess portion of the wire can be cut and removed.

For simplicity, 3A and 3B show the implant 50 in a linear configuration. However, for annuloplasty procedures, the implant 50 can generally be implanted along a curved path (or even a complete ring) around the valve annulus, such that the contraction/tensioning of the wire/line 58 reduces the size (e.g., a radius, etc.) of the valve annulus, improving coaptation of the valve leaflets.

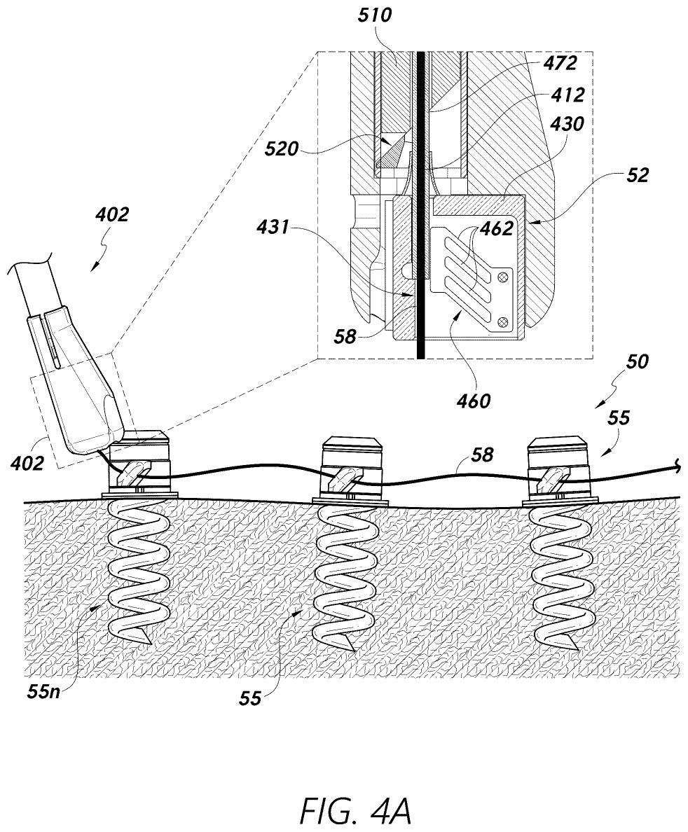

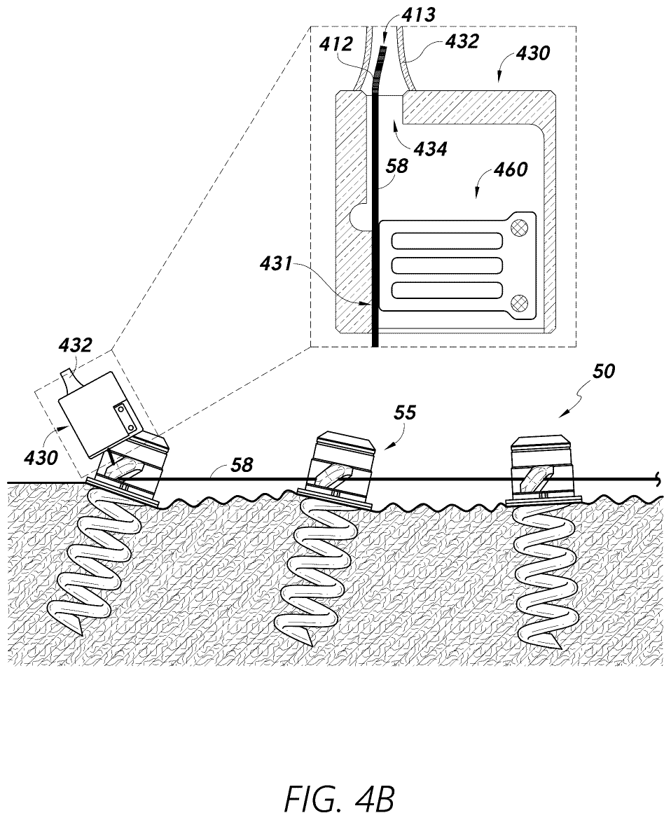

FIGS. 4A and 4B show portions of an example tissue anchor implant assembly 50 and tension locking and/or cutting system 402 in accordance with one or more implementations. In some implementations, the tension locking and cutting system 402 is coupled to the tensioning wire/line 58 in the vicinity of a terminal tissue anchor 55n of the plurality of anchors 55 of the implant 50.

In some implementations, the tension locking and cutting system includes a line cover device 52, which comprises one or more fasteners 460 configured to lock the wire/line 58 in place relative to the line cover device 52 and/or terminal anchor 55n, thereby locking a tension in the wire/line 58. In some implementations, the line cover device 52 does not comprise fastener(s), in which case the wire/line 58 can be tension-locked by a fastener/locking mechanism/means that is separate and discrete from the line cover device.

In some implementations, the line cover 52 includes a line-covering projection 432, which can comprise cloth in some implementations and covers an excess/end portion of the wire/line 58. In some implementations, the line cover device 52 is configured to cover the excess portions 412 of the wire/line 58 proximal to the terminal anchor 55n by drawing the excess portions 412 of the wire/line 58 within a housing 430 of the line cover device 52.

Once the wire/line 58 is clipped or cut following cinching of the implant 50 in order to perform annuloplasty, it can be advantageous to cover a free end 413 of the wire/line 58. Covering of the free end 413 of the wire 58 can help prevent damage to tissue that can be caused by exposure of the metal/tip of the wire 58. Additionally, covering of the free end 413 and excess portions of the wire/line 58 can prevent additional fibrosis around such wire portions. In some implementations, the wire/line 58 is advantageously comprised of metal since metal is generally stronger and more durable than certain fabric sutures, for example. Additionally, where the wire/line 58 comprises conductive material, it can be viewed under fluoroscopy during the annuloplasty procedure.

In some implementations, once all the tissue anchors 55 have been implanted, the wire/line 58 can be threaded through the line cover device and line-cutting tool 402, which can be advanced along the wire/line 58 to the anchor 55n. The relative spatial orientation of the components of tool 402 can enable the wire 58 to pass straightly and directly though the lumen of the tool 402 in a direct and unwinding path that reduces friction on the wire/line 58 as it moves within the tool 402.

As referenced above, the housing 430 of the line cover device 52 can house, at least in part, a line fastener 460, which can function as a tension locking mechanism. In some implementations, the fastener 460 is shaped so as to define a generally-rectangular, planar clip comprising a super-elastic material, such as nitinol or the like. The fastener 460 advantageously can comprise a deformable element shaped so as to define a plurality of slits which are surrounded by a plurality of flexible struts 462 which enable the clip to transition between slanted (see FIG. 4A) and straight (see FIG. 4B) states/configurations.

In some implementations, the line-engaging surface of the clip 460 can be formed to define a plurality of teeth (e.g., jagged teeth; not shown for clarity) configured to increase friction between the wire/line 58, 412 and the fastener 460. It is to be noted that the particular illustrated fastener design 460 in FIGS. 4A and 4B is represented by way of illustration and not limitation and that any suitable securing means, fastener, clip, etc. can be used.

In some implementations, the fastener 460 provides a clamping structure that is biased toward a closed state/configuration (see FIG. 4B). In the closed state/configuration, the clamping structure is configured to clamp onto the wire 412 passed therethrough/thereby. In some implementations, the fastener 460 can be flexed to an open state/configuration (see FIG. 4A) configured to allow for movement of the wire 412 through the cover device 52. Conversely, in the closed state, the fastener 460 is configured to restrict movement of the wire portion 412 with respect to the terminal anchor 55n and/or the cover 52.

In some implementations, the wire/line portion 412 extends through an opening 434 in the housing 430 and/or through a line-covering projection 432 that projects from the opening 434 in the housing 430. In some implementations, the excess portion 412 of the wire/line can extend through an interference tube 472 that can define a lumen therethrough for surrounding at least a portion of the wire 412.

In some implementations, the interference tube 472 can be removable from the housing 430 of the cover 52. In some implementations, the interference tube 472 can be shaped so as to fit snugly within a channel extending from the opening 434 such that it pushes against the line-engaging surface of the clip/fastener 460 and maintains the clip/fastener 460 in the slanted/unlocked state shown in FIG. 4A.

In some implementations, in the slanted state/configuration, the clip/fastener 460 is deformed and does not push against the wire 412, but rather pushes against the interference tube 472. In the slanted state/configuration, the wire 412 is free to move with respect to the fastener 460, housing 430, and/or interference tube 472. In such a configuration, the wire/line 58, 412 can be pulled until it sufficiently contracts the annuloplasty implant 50.

In FIG. 4B, the interference tube 472 has been decoupled and removed from the housing 430. In the absence of force applied to the line-engaging surface of the clip/fastener 460 by the interference tube 472, the bias of the clip 460 cause the clip 460 to return to its resting, straight state/configuration, wherein in such state, the clip/fastener 460 effectively traps the wire 412 between the line-engaging surface of the clip 460 and a surface 431 of the housing 430 (e.g., an inner wall). As such, the fastener 460 can assume a locked configuration in which the clip/fastener 460 locks and crimps the wire 412 to the housing 430.

In some implementations, the line cover device and line-cutting tool 402 can comprise a static cutting element 510 and a moveable, dynamic cutting element 520, each of such cutting elements defining a sharp edge. In some implementations, the dynamic cutting element 520 can be configured to slide proximally and diagonally with respect to the static cutting element 510.

As described above, in some implementations, once the interference tube 472 is displaced from within the housing 430, the fastener/clip 460 can assume the closed position in order to trap the wire 58 between the clamping surface of fastener 460 and the surface 431 of the housing 430. In some implementations, such displacement of the interference tube 472 can also enable the interference tube 472 to push (e.g., by hammering) proximally on the dynamic cutting element 520 such that it slides proximally diagonally along the static cutting element 510 in a manner in which the elements 510 and 520 sever and cut the wire/line 58. Thus, the tool 402 can advantageously provide a mechanism that enables contemporaneous cutting and locking of the wire 58. Thus, the tool 402 can be arranged such that it advantageously provides a safety mechanism by which the wire 58 can only be severed by proximal force applied thereto by the interference tube 472 after the fastener 460 has been transitioned into the fastened/locked state in which the tension in the wire 58 is substantially fixed. That is, the tool 402 can be configured such that it cannot inadvertently sever the wire 58 while the tool 402 is not coupled to the interference tube 472 and while the interference tube 472 does not push against the cutting element 520.

In some implementations, the line cover device 52 comprises a flap/cap (not shown) that is moveable from an open state to a closed state in which the flap/cap pushes any excess portions of the wire 58 exiting the housing 430 via the opening 434 against an external surface of the housing 430 in order to fix the wire 58 to the housing 430 and/or seal/cover the opening 434. In some implementations, the housing 430 is sealed/covered over the end 413 of the wire/line 58 by the proximal projection 432, which can comprise braided fabric mesh or other material. Such material can be configured to collapse on the wire end 413 in some implementations.

### Tensioning Devices and Systems

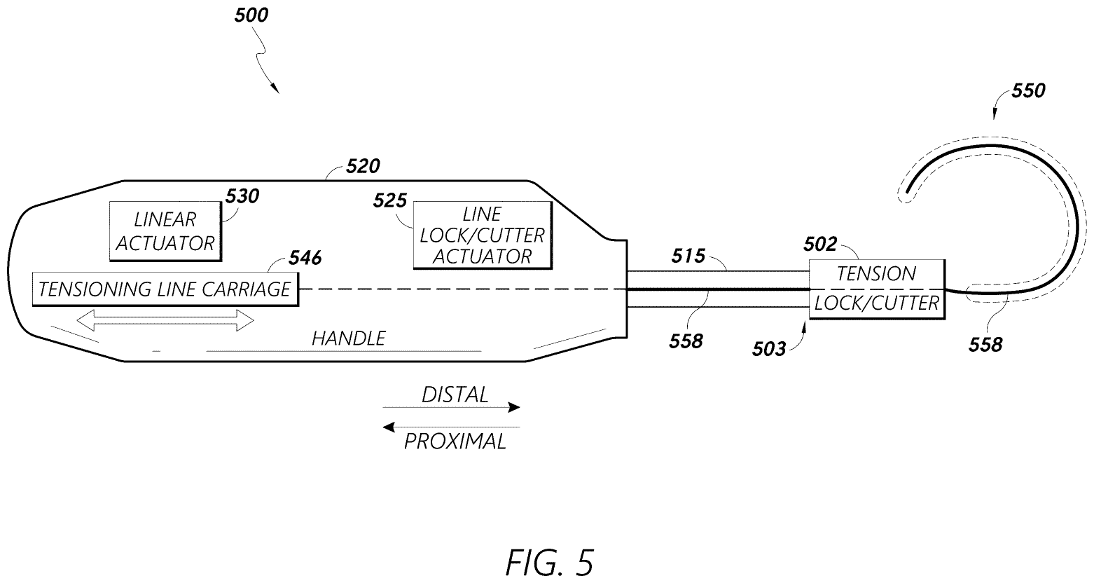

FIG. 5 shows an example implant tensioning system 500 in accordance with one or more implementations. In some implementations, the tensioning system 500 includes a tensioning device 520, which can take the form of a handle and can be manipulable by a user when in connection with execution of tensioning adjustment with respect to an implant device. Although handles are described herein as examples of tensioning devices and/or components of tensioning systems, it should be understood that such devices can have any suitable form or configuration. In some implementations, the tensioning system 500 can be employed in connection with implantation of an annuloplasty or other type of implant device 550 having a tensioning line/wire 558 associated therewith.

After implant deployment, the tensioning device/system 500 can be utilized in order to contract the implant 550 to bring the target tissue (e.g., native valve annulus) together and/or into a desired reshaping. For example, for tissue-anchor-based annuloplasty implant devices as described in detail herein, after the last tissue anchor of the annuloplasty implant is deployed, the implant 550 can be disconnected from an anchor driver system/device used to implant/embed the implant tissue anchor(s), and the anchor driver system/device can be subsequently removed.

In some implementations, the tensioning device/system 500 can then be advanced over the implant tensioning wire 558. For example, the proximal portion of the tensioning line 558 can be drawn through a tubular member 515 extending distally from the handle 520, and further through at least a portion of the handle 520, where it can be fixed in some manner to a linearly-actuatable tensioning line carriage 546. In some implementations, the tensioning line carriage 546 can be actuatable using a linear actuator component 530 (e.g., manually rotatable knob) associated with the handle 520.

In some implementations, the distal end 503 of the tubular member 515 can be advanced into proximity or contact with the last anchor, and/or the distal end, of the implant 550. In some implementations, a tension line locking and/or cutting device/member 502 can be disposed at a proximal end of the implant 550 and/or distal end of the tubular member 515. In some implementations, the device 502 can be controllable through engagement with certain actuator component(s) 525 configured to cause locking and/or cutting of the tensioned line 558 at the line lock/cutter device(s)/component(s) 502. Although the functional components 525, 502 are shows as single boxes, it should be understood that such components can be embodied in multiple separate/discrete components in some implementations.

In some implementations, the tubular member 515 can advantageously be non-compressible, or minimally compressible, in the axial dimension, which can advantageously maintain a fixed or set distance between the distal end of the handle 520 and the implant device 550, thereby facilitating tension adjustment by linearly translating the tension line 558 at the handle 520 without significant losses due to axial compression. Furthermore, use of the tubular member 515 can allow for the advancement of the device 500 through the anatomy of the patient (e.g., within an access sheath) to the implantation site. The tube 515 can comprise any suitable or desirable material and/or specifications sufficient to provide the necessary or desirable axial stability and flexibility.

In some implementations, after the tensioning line/wire 558 has been advanced/passed through/into the tensioning device/handle 520, contraction of the implant can be implemented by rotating or otherwise manipulating the linear actuator 530 (e.g., an adjustment roller/knob, or the like) to thereby draw the tensioning wire 558 proximally such that the tissue anchors of the implant 550, which are each coupled to the line 558, are brought relatively closer together to form the desired configuration of the implant device 550. The tensioning device/handle 520 and/or linear actuator 530 can be operated by hand to adjust tension in the tensioning line 558 according to some solutions.

In some implementations, when operating the system 500, adequate reduction of mitral regurgitation or other condition severity can be assessed, such as by using echocardiography or other imaging system under beating-heart conditions. When the appropriate implant size/shape has been achieved, the tensioning device 520 can be detached from the implant device 550 and/or withdrawn from the tensioning wire 558, thereby leaving the contracted implant 550 deployed in the target anatomy with the desired degree of contraction.

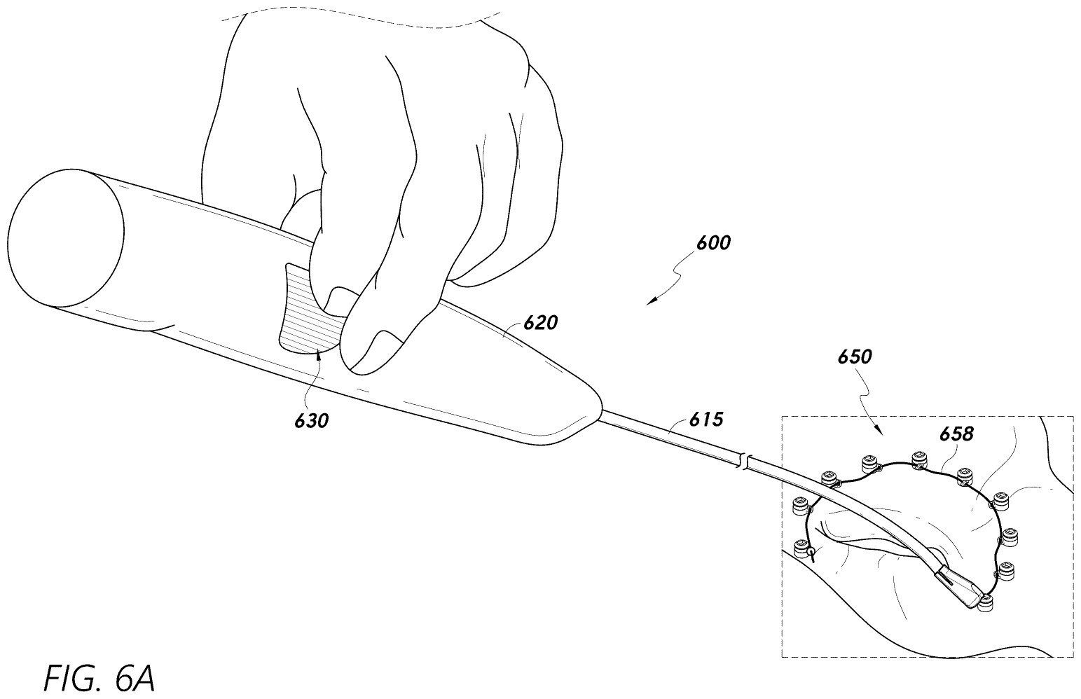

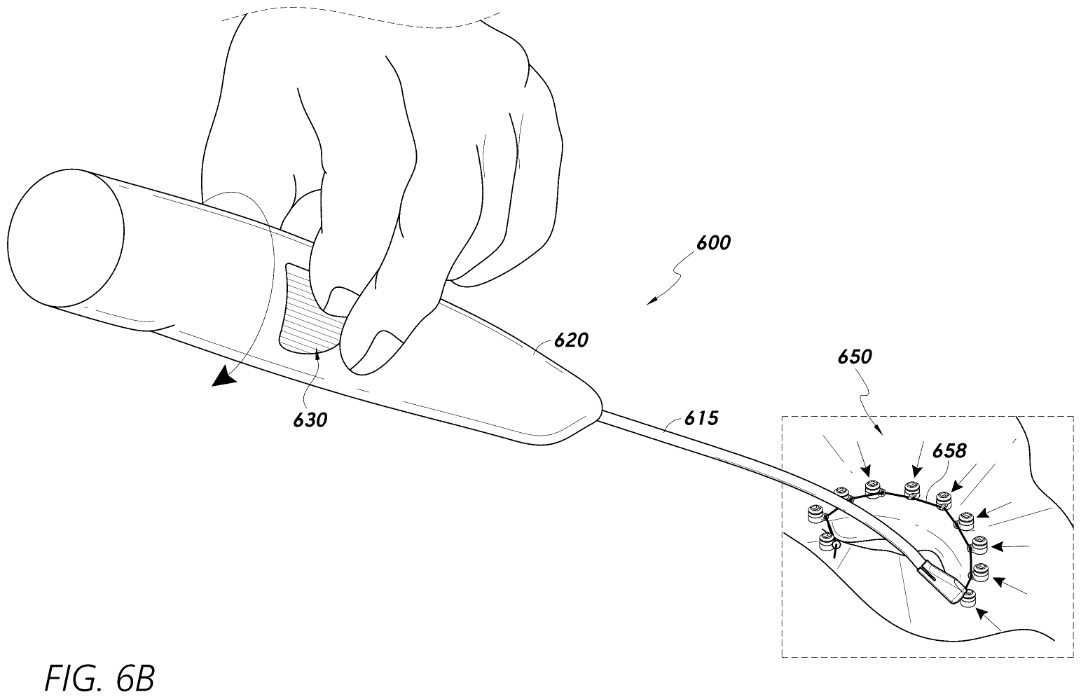

FIGS. 6A and 6B show stages of an implant tensioning process that can be implemented to tension a tensioning line 658 associated with an annuloplasty implant device using a tensioning device/system 600 by manually manipulating a linear actuator knob 630 in accordance with one or more implementations. The actuator knob 630 can be associated with a handle component 630 of the system 600.

In some implementations, the tensioning device 600, which can be considered a size adjustment tool with respect to the size (e.g., radius, length, etc.) of the implant device 650, can be introduced over the tensioning line/wire 658. Once the tensioning line/wire 658 has been passed into/through the handle 620 and/or fixed to a portion thereof (e.g., a portion of a tension wire carriage configured to be linearly/axially actuated), the linear actuator adjustment knob 630 can be rotated (e.g., clockwise or counterclockwise rotation about an axis of the handle 620 and/or tube 614) to effect implant contraction.

Generally, for some solutions, adjustments of the tension of an implant tether/line can be made through manual rotation of an adjustment knob, such as the knob 630. The amount of line tension can be understood to be non-linear with respect to the amount of tensioning force applied thereto. Therefore, for certain manual tensioning solutions, the human operator may be prone to over-tensioning of the relevant line, thereby potentially resulting in undesired physiological effects. In view of such considerations, the threading of the rotatable actuator 630 and/or internal tension line carriage component(s) can be designed to facilitate relatively fine tensioning.

FIG. 6A shows the annuloplasty implant 650 in an initial state after anchoring of the implant 650 to the target tissue 10, but prior to tensioning of the line/wire 658, and therefore prior to correction/remodeling of the native valve annulus 10. As shown in FIG. 6B, after some amount of manual rotation of the actuator 630 at the handle 620 by the operator, the tension wire 658 can become tightened, such that the distance thereof disposed between ends of the implant device 650 is reduced, thereby causing the implant 650 to contract and produce a shape having a reduced radius/diameter.

Various tensioning and un-tensioning steps/periods can be implemented during the tensioning process. Depending on the pitch of the relevant threads of the linear actuator 630 and/or tension wire carriage associated with the handle 620, the operator may be subject to significant and/or undesirable strain and/or physical activity, and furthermore significant time may be required, in order to effect the amount of tensioning needed or desired in the tensioning wire 658. In particular, where relatively substantial tensioning or un-tensioning of the tension wire 658 may be desired or required as a means to correct improper tensioning and/or to more quickly achieve a desired tensioning level for the tensioning line 658, the time and stress/energy burden associated with rotation of the knob 630 by the operator can be undesirable or problematic.

Implementations of the present disclosure provide simplified means for achieving desired tension adjustment. For example, some implementations include actuator components that allow for quick release of the threaded engagement thereof with the tension wire carriage (and/or other component(s) configured to directly and/or indirectly actuate, or cause actuation of, the carriage) to allow for gross manual adjustment of the wire tension by manually sliding/translating the tension wire carriage, rather than requiring tedious knob rotation for relatively larger steps in tension adjustment. Furthermore, some implementations provide for motorized tensioning adjustment, wherein the rate of tensioning adjustment and/or mechanisms associated therewith can provide for reduced time and/or physical strain in connection with tensioning operations.

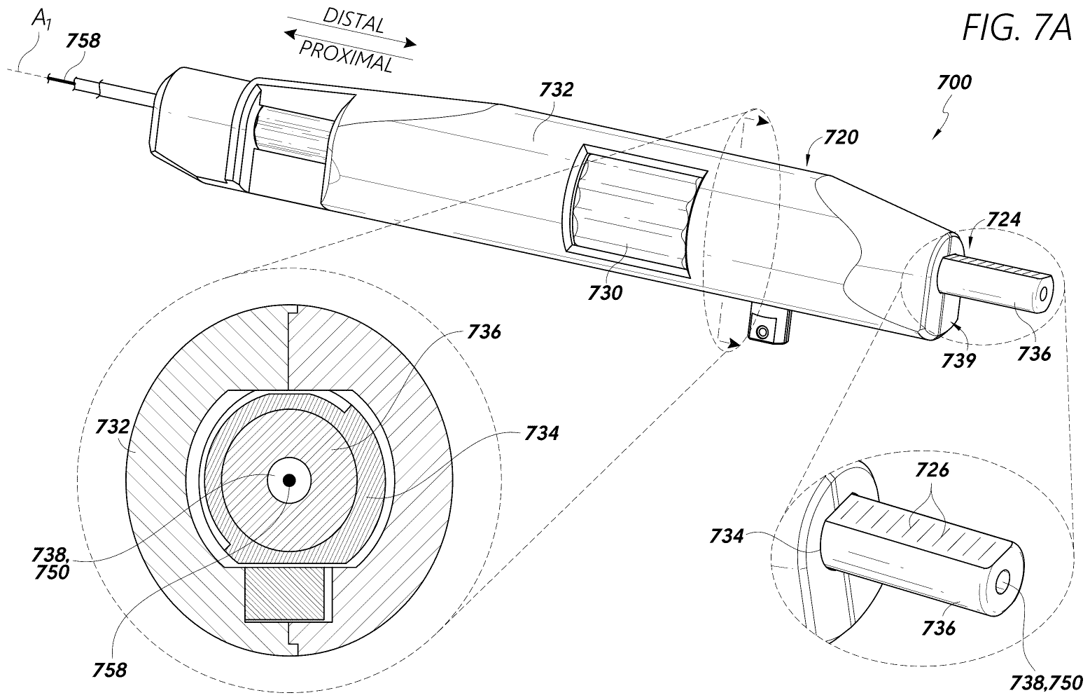

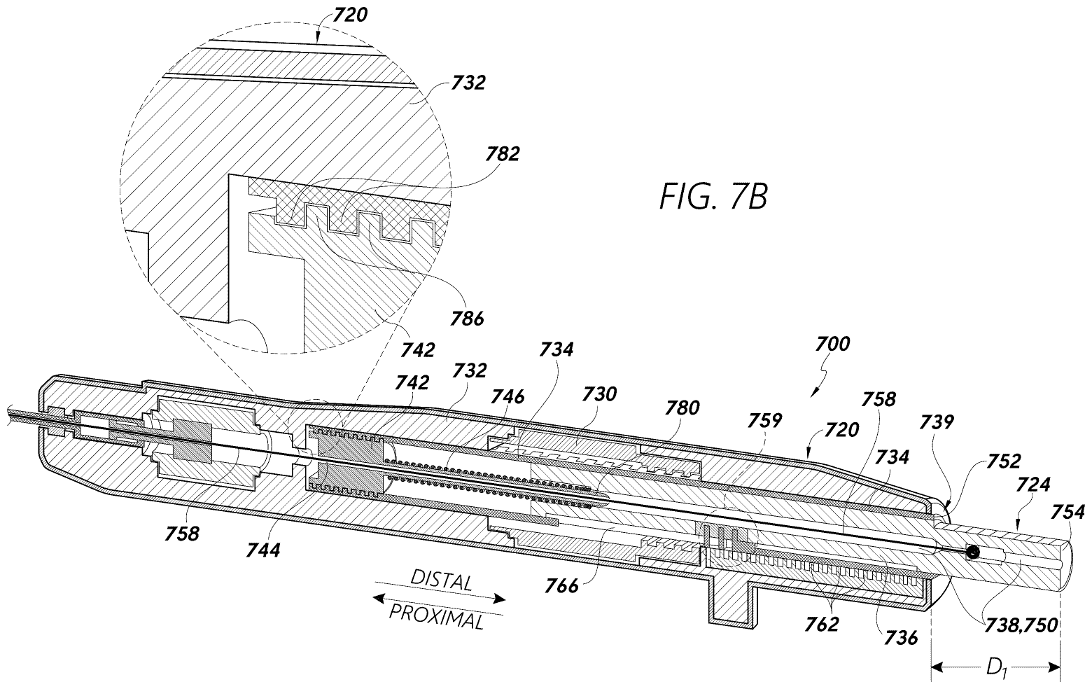

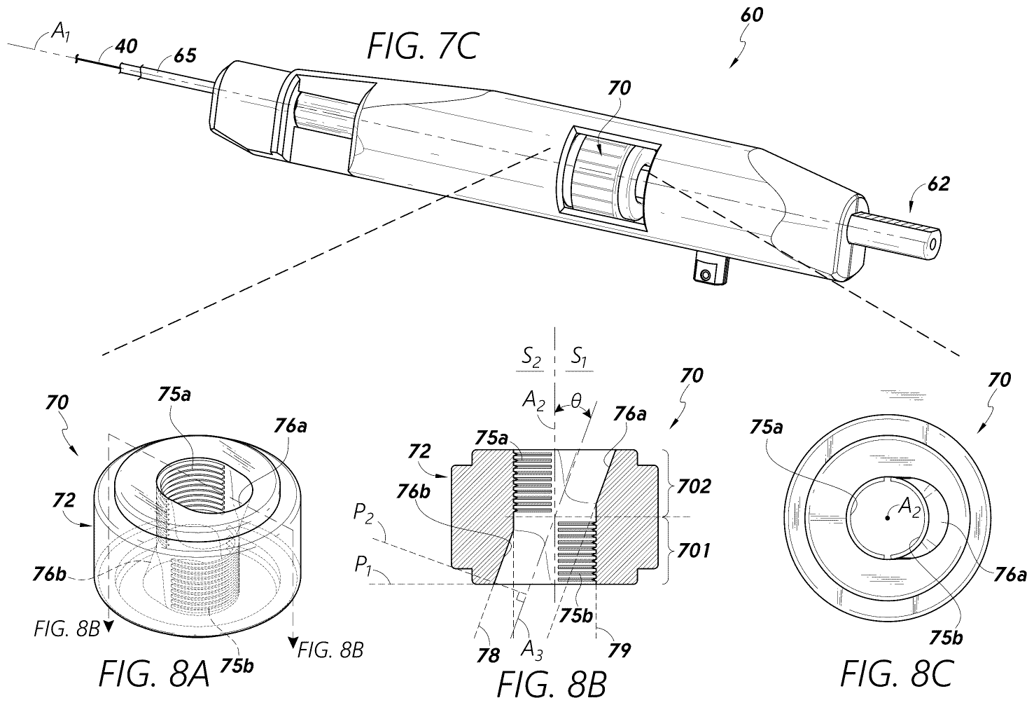

FIGS. 7A, 7B, and 7C provide schematic illustrations of an example tensioning device 700 in accordance with one or more implementations. In some implementations, the tensioning device 700 is configured to contract and loosen an implant-coupling line/wire 758, as described in detail herein.

In some implementations, the tensioning device 700 can be used with an implant comprising an implantable structure and a flexible elongated contracting/coupling line 758 that extends from the implantable structure. The implant can comprise any of the implants described herein, such as implantable annuloplasty structures, which can, for example, comprise a plurality of tissue anchors coupled to the coupling/tensioning line 758.

In some implementations, the tensioning device 700 comprises a handle portion 720, which optionally can be supported by a stand in some implementations. The handle 720 can comprise an outer housing 732, which can be shaped ergonomically for holding by a user (e.g., a physician, healthcare technician, etc.). The handle 720 can further comprise a tubular shaft 734, disposed at least partially within outer housing 732.

In some implementations, an inner rod 736 can be at least partially disposed within a proximal portion of the tubular shaft 734, such that the inner rod 736 is axially slidable with respect to the tubular shaft 734. In some implementations, the inner rod 736 can be associated with and/or part of a tenson line/wire carriage component 736.

In some implementations, the rod 736 can be shaped so as to define a channel 738 configured to receive the tensioning line 758 therein; the tensioning line 758 can be secured/fixed to the rod/carriage 736 in some implementations.

In some implementations, the handle 720 can include a distal force applicator 742, which can be disposed at least partially within a distal portion of the shaft 734, and can define a channel 744 configured to receive, and allow sliding of, the tension line/wire 758 therethrough. In some implementations, a spring 746 can be disposed within the shaft 734, which can connect the distal force applicator 742 to a distal portion of the rod 736.

In some implementations, the handle 720 further comprises an actuator knob 730, which can be configured to cause linear/axial actuation of the carriage rod 736, thereby changing the tension in the tension line/wire 758. In some implementations, the actuator knob 730 can be accessible from outside outer housing 732 to allow for manual manipulation thereof to effect tensioning of the line 758.

Although various internal components of the handle 720 are illustrated and described herein, it should be understood that any internal components can be utilized/implemented in connection with line tensioning and actuation of associated components; it should be understood that tensioning handle devices disclosed herein in connection with implementations of the present disclosure can generally include actuator knobs/mechanisms that are configured to be rotated or otherwise manipulated to cause axial/linear movement of a carriage or other component fixed to a tensioning line to thereby cause tensioning and/or untensioning of the line, regardless of the particular mechanical components utilized. Such linear actuation, in some implementations, is effected through engagement between threads associated with the manipulatable knob/actuator and threads associated with one or more internal components configured to cause linear movement of the tensioning line.

In some implementations, the handle 720 can include a channel 750 from a distal end through to a proximal end of thereof. In some implementations, the channel 750 can include an inner line/wire channel 738, as well as a distal-force-applicator line/wire channel 744.

In some implementations, a portion of the tension line/wire 758 can be threaded through the channel 750 either after or before the implantable structure and line/wire 758 are advanced toward the heart of the patient.

In some implementations, the handle 720 can be configured such that actuation of the tensioning actuator knob 730 in a tightening direction, when the line/wire 758 is disposed within the channel 750, causes the handle 720 to proximally uptake the line/wire 758. Before initial actuation of the actuator knob 730, the portion of the line/wire 758 between handle portion 720 and the implant can be somewhat slack or minimally tensed. In some implementations, a proximal end 752 of the tubular shaft 734 and a proximal end 754 of the inner rod/carriage 736 are disposed at an offset distance D₁, therebetween, which can indicate an amount of tension in the line/wire 758.

In some implementations, the actuation of the tensioning knob 730 can cause the handle 720 to linearly actuate the line/wire carriage 736, which is fixed directly or indirectly to the line/wire 758. For example, such tensioning can be implemented by causing linear/axial translation of the tubular shaft 734 proximally with respect to the outer housing 732, which can cause the distal force applicator 742 to advance proximally with respect to the outer housing 732; the distal force applicator 742 can be axially fixed to the tubular shaft 734 during ordinary use of the handle 720.

In some implementations, proximal axial translation of the force applicator 742 can apply a proximally-directed force to the spring 746, which pushes the line/wire carriage 736 proximally with respect to the outer housing 732 (by spring 746 applying a proximally-directed force to the carriage 736, which proximally pulls the line/wire 758).

In some implementations, when the line/wire 758 is relatively slack, the tension force on the carriage 736 from fixation to the line/wire 758 may offer relatively little resistance to the proximally-directed force applied to carriage 736 by the spring 746. As the knob/actuator 730 is actuated, the carriage 736 moves proximally, thereby increasing the tension in the line 758, and the carriage 736 gradually offers increasing resistance to the proximally-directed force applied to the carriage 736 by the spring 746, and spring 746 becomes gradually more compressed.

In some implementations, as the spring 746 becomes more compressed, the distal force applicator 742 moves axially closer to carriage 736, such that the tubular shaft 734 moves proximally with respect to the carriage 736. As a result, the spring 746 pushes the carriage 736 proximally with respect to the outer housing 732 to a lesser extent than the tubular shaft 734 proximally advances with respect to the outer housing 732, and proximal pulling of the line/wire 758 by the carriage 736 increases tension in the line/wire 758. Therefore, the offset distance between the proximal end 752 of the tubular shaft 734 and the proximal end 754 of the carriage 736 decreases, as the portion of the carriage 736 that protrudes from the proximal end 752 of the tubular shaft 734 has decreased.

The tensioning knob/actuator 730 can have any shape that enables actuation thereof, and is not necessarily round, tubular, or generally cylindrical. For example, in some implementations, the tensioning knob 730 can be configured to be actuated by rotation thereof, such as about a central longitudinal axis A₁, of the tubular shaft 734. In some implementations, the tensioning actuator/knob 730 is configured to be actuated by axially sliding the actuator/knob with respect to the outer housing 732. The tensioning actuator/knob 730 can be entirely mechanical, or can optionally comprise electrical components, including, for example, circuitry.

In some implementations, the tubular shaft 734 and the tensioning knob 730 are in threaded connection with each other, and the handle portion 720 is configured such that actuation of tensioning knob 730 rotates the tubular shaft 734, thereby advancing the tubular shaft 734 proximally with respect to the outer housing 732. For example, the tensioning knob 730 can be configured to be actuated by rotation thereof, such as about the central longitudinal axis A₁ of the tubular shaft 734.

In some implementations, the handle assembly 720 further comprises a translation rod 780. References herein to a line/wire carriage may be understood to refer to a carriage bar (e.g., carriage bar 736), a translation rod (e.g., translation rod 780; can be threaded for engagement with a nut or gear, or can be associated/coupled with a threaded member (e.g., distal force applicator 742) configured to engage with a nut/gear), or both.

In some implementations, the translation rod 780 is configured to translate axially in response to actuation of the actuator 730. The translation rod 780 can extend proximally from, and/or be axially fixed to, the distal force applicator 742, and can define therethrough a portion of the line/wire channel 750.

In some implementations, a portion of the translation rod 780 can be disposed within the channel 738; the length of the portion can vary with the distance between distal force applicator 742 and the carriage bar 736. The spring 746 can be configured to surround at least a portion of the translation rod 780 and be free to move axially with respect to the outer surface of the translation rod 780.

In some implementations, the carriage bar 736 partially protrudes out of a proximal end 739 of the outer housing 732, such that a portion of the carriage 736 is visible to the user. For example, the tubular shaft 734 and the carriage 736 together can provide a non-electrical mechanical force gauge 724, in which a relative axial position of the tubular shaft 734 with respect to the carriage bar 736 and/or translation rod 780 (i.e., the offset distance D₁, between the proximal end 752 of the tubular shaft 734 and the proximal end 754 of the carriage bar 736) provides a visual indication of a measure of the tension in the line/wire 758. In some implementations, the tubular shaft 734, at least after it begins advancing proximally, can also protrude out of the proximal end 739 of outer housing 732. For example, the carriage 736 can be marked with a plurality of fiducial markers 726, which can be arranged along a surface of the carriage bar 736 to indicate the relative axial position of the tubular shaft 734 with respect to the carriage 736.

It is noted that the force gauge 724 generally may not measure the length of the line/wire 758 that the handle portion 720 uptakes, which is generally equal to the distance that the carriage 736 moves proximally. For example, as discussed above, an initial portion of the uptake length is sometimes due to proximal movement of the carriage 736 while tubular shaft 734 proximally moves approximately in tandem with the carriage 736 before the line/wire 758 is tensioned. During this optional initial motion, tension in the line/wire 758 may not materially increase, even though handle portion 720 uptakes some length of the line/wire 758.

In some implementations, the carriage 736 does not protrude out of proximal end 739 of the outer housing 732, in which case the handle 720 does not provide a non-electrical mechanical force gauge. The handle 720 may nevertheless still be entirely useful for regulating the tension in the line/wire 758, such as is configurations in which handle 720 further comprises a tension-limiting locking assembly 759 for limiting the maximum tension that the carriage 736 can apply to the line/wire 758. For example, the tension-limiting assembly 759 can be configured to axially lock the carriage 736 relative to the outer housing 732 when the tension in the line/wire 758 reaches a predetermined threshold level, thereby limiting the maximum tension that the carriage 736 can apply to line/wire 758.

In some implementations, the tension-limiting locking assembly 759 can be configured to axially lock the carriage 736 with respect to outer housing 732 and/or to axially lock the tubular shaft 734 with respect to outer housing 732 when the tubular shaft 734 is disposed at a predetermined relative axial position with respect to carriage 736. In some implementations, the tension-limiting locking assembly 758 comprises a detent that is arranged to axially lock the carriage 736 with respect to the outer housing 732 when the tubular shaft 734 is disposed at the predetermined relative axial position with respect to the carriage 736, thereby limiting the maximum tension that the carriage 736 can apply to the line/wire 758. In some implementations, the predetermined relative axial position of the tubular shaft 734 with respect to the carriage 736 can have the effect of setting a predetermined maximum tension that can be applied to line/wire 758 using the tensioning device 700.

In some implementations, as the spring 746 becomes more compressed, the distal force applicator 742 moves axially closer to carriage 736, such that the tubular shaft 734 moves proximally with respect to the carriage 736. As a result, the offset distance D₁ between the proximal end 752 of the tubular shaft 734 and the proximal end 754 of the carriage 736 decreases.

In some implementations, the distally-directed motion of the tubular shaft 734 can be caused by actuation of tensioning knob 730 in the opposite direction of actuation for the proximally-directed motion described hereinabove. Such operation can allow the user to reduce the tension in the line/wire 758 if necessary during the procedure. In some implementations, once the desired level of tension in the line/wire 758 is achieved (by monitoring force gauge 724, by tension-limiting mechanism 759, and/or, for example, by monitoring the extent of regurgitation of the valve under echocardiographic and/or fluoroscopic guidance), the tensioning tool 700 can lock the line/wire 758 so as to maintain a degree of tension in line/wire 758 in order to maintain the implant (not shown in FIGS. 7A-7B) coupled to the system 700 in a contracted state.

A radially-inward surface of the tubular shaft 734 near a distal end thereof can be shaped so as to define threading 782. A radially-outward surface of the distal force applicator 742 can be shaped so as to define a corresponding threading 786. The threads can allow for fine tuning of the axial location of the carriage 736 relative to the shaft 734, by effecting fine rotation of the distal force applicator 742 with respect to the tubular shaft 734. During use of the handle 720 during a medical procedure, as described hereinabove, the distal force applicator 742 is rotationally fixed, and thus axially fixed, with respect to tubular shaft 734. In some implementations, the adjustment of the axial location of distal force applicator 742 with respect to the tubular shaft 734 during the calibration procedure adjusts the preload in the spring 746 (by compression of the spring) to set a desired level of maximum tension that carriage 736 can apply to line/wire 758.

### Quick-Release Actuator Knob

Implementations of the present disclosure provide tensioning devices and tensioning device handles configured to allow for relatively simplified gross- and fine-tuning of implant line/wire tension. That is, implementations of the present disclosure provide mechanisms for toggling or otherwise selectively transitioning between gross-tuning and fine-tuning configurations of a tensioning device and/or tensioning actuator.

With respect to certain tensioning actuators, the gear ratio implemented in the threads utilized for linear actuation can be configured to facilitate ease of rotation of the actuator/knob, stability, and/or the ability to fine-tune the tension of the tensioning line/wire. However, in some instances, it may be necessary or desirable to perform relatively large adjustments in tension of the tensioning wire, as opposed to the relatively finer actuation of threaded actuator knobs. For example, in early stages of tensioning and/or in instances in which de-tensioning and/or re-tensioning are required or desired, such tensioning may be better suited for gross tensioning in a manner that does not require the operator to manually rotate the actuator knob. For example, in some instances, a relatively large number of rotations of the actuator may be necessary in order to achieve the desired gross-tensioning adjustment, which can be tiring for the operator and/or time-consuming.

Some implementations of the present disclosure provide for actuator knobs that provide a quick-release mechanism for selectively transitioning the actuator from a thread-meshed, fine-tuning tensioning engagement/configuration to a non-thread-meshed, gross-tensioning configuration, and/or vice versa.

FIGS. 8A-8C show perspective, side cross-sectional, and axial views, respectively, of an example tensioning actuator 70 in accordance with one or more implementations. The tensioning actuator 70 can be configured to allow for gross- and fine-tuning of a tensioning line/tether 40, which can be associated with an annuloplasty implant device or other device or system. For example, as described above, in order to provide fine-tuning of tension in tensioning wires/lines using a tensioning device as described in detail above (e.g., tensioning tool 700 shown in FIGS. 7A-7C), a rotation-based linear actuator can be utilized, wherein rotation of the actuator about an axis produces axial movement of a carriage or other component(s)/structure(s) directly or indirectly fixed to a tensioning line/wire, thereby allowing for tension in the line to be modified through clockwise and/or counterclockwise rotation of the actuator.

In some implementations, the tensioning actuator 70 can be configured to engage threads of a tensioning wire carriage 62, or threads of any other bolt-type structure directly or indirectly coupled or associated with the carriage 62 in a manner as to affect linear actuation of the carriage 62, in a fine-tuning configuration, wherein in such configuration, the actuator 70 can operate in a similar manner as the actuator 730 described above in connection with FIGS. 7A and 7B in one or more respects.

Description herein of an actuator engaging with threads of a bolt-type structure/component should be understood to relate to the threaded/mesh engagement of actuator threads with corresponding threads of any structure fixed, directly or indirectly, to a tensioning line/wire, such that axial translation of the structure/component causes axial translation (e.g., pulling, letting) of the tensioning line/wire relative to a handle or other component associated with the actuator.

In some implementations, the actuator/knob 70 can further be configured to be manually disengaged and/or manually adjusted/manipulated to a disengaged configuration, such that internal threads 75 of the actuator 70 are released from engagement with the corresponding threads of the axially translatable bolt (e.g., carriage 62 and/or component of associated assembly), thereby allowing for the bolt to slide relatively freely/within (e.g., along an axis of the bolt) the handle 60.

In some implementations, the disengaged configuration of the actuator 70 can be tilted by an amount θ relative to the axis A₂ of a threaded pseudo channel 79 of the actuator 70 associated with the engaged configuration. In the disengaged configuration, the operator can be able to make gross adjustments to the tension of the tensioning line 40 simply by manually axially sliding the bolt/carriage 62 without utilizing the threading of the actuator 70 or the carriage 62 and/or associated component(s). In some implementations, the threaded pseudo channel 79 (or the pseudo channel 78 in implementations in which the threaded pseudo channel 79 is the one that is angled) is parallel and/or aligned with the axis A₂ of the actuator 70, as shown.

In some implementations, the tensioning actuator 70 can be threaded in a manner such that an operator can be permitted to rotate the actuator/knob 70 such that a threaded arrangement of the actuator 70 with mating/meshing threads of a tensioning wire carriage component draws the threaded carriage/bolt axially with respect to an axis A₁ of the tensioning device handle 60.

In some implementations, the threaded tensioning wire carriage/bolt 62 can be fixedly attached to the implant line/tether 40, as described above. Therefore, axial movement of the carriage/bolt 62 relative to the handle 60 and/or implant device can result in tension adjustment with respect to the tensioning line 40.

In some implementations, an axially incompressible/rigid tube 65 can extend between the handle 60 and the implant (not shown in FIGS. 8A-8C), such that the tube 65 maintains a fixed distance of the line/wire 40 between the distal end of the handle 60 and the implant. Therefore, axial movement of the carriage/bolt 62 relative to the handle 60 and/or tube 65 can result in tightening of the tensioning wire/tether at the implant.

In some implementations, the tilt-based quick-release nut/actuator 70 includes inner diameter surfaces 75, 76 that conform to two separate relatively angularly-offset/tilted pseudo channels 78, 79, wherein the two tilt positions of the nut/actuator 70 relative to the axis A₁ of the handle 60 alternatingly align the channels 78, 79 with the axis A₁, of the line carriage 62.

In some implementations, when the threaded channel 79 is aligned with the carriage 62, the actuator 70 is threadingly engaged/meshed with the threads of the bolt structure, wherein tilting of the actuator 70 causes the threads 75 to become disengaged from the threads of the bolt to allow for free axial sliding of the carriage 62. Although the threaded channel 79 is shown as orthogonal to the transverse plane P₁ of the actuator 70, whereas the non-threaded channel 78 is shown as angled relative to the plane P₁, it should be understood that in some implementations, the non-threaded channel 78 can be orthogonal to the plane P₁, whereas the threaded channel 79 can be angled thereto.

In some implementations, the angularly-offset pseudo-channels 78, 79 intersect, as shown, such that they are partially merged to form an irregular bi-conically-tapered channel/cavity within the actuator 70. For example, the merging of the angled 78 and axial 79 channels can produce flared openings open to both axial sides of the actuator 70, as shown. In some implementations, tilting of the actuator by an amount θ can align the smooth inner surfaces 76 associated with the pseudo-channel 78, which is aligned with and/or defined by the smooth surfaces 76, with the axis A₁ of the wire tension carriage 62. In such configuration, the threads of the carriage 62 or other bolt member can be permitted to slide relative to the inner diameter/surface of the actuator 70 without getting caught in the threads 75. In some implementations, the threads of the carriage 62 can contact and slide against the smooth surfaces 76 of the inner diameter of the channel 78 in the gross-tension adjustment configuration.

In some implementations, the threads 75a, 75b may run along only a partial circumferential portion of the inner diameter of the channel 79. For example, the threading 75 may run only along a portion of the merged channels corresponding to the pseudo channel 79 and not along the walls corresponding to the pseudo channel 78. That is, half or less of the circumference of the inner diameter of the merged channel corresponding to the axial channel 79 can be threaded, whereas the remainder of the circumference can be smooth.

In some implementations, the threading 75a and 75b can be associated with separate axial portions (e.g., halves) 701, 702 of the channel 79, and can advantageously be non-overlapping circumferentially to facilitate disengagement with the threading 75 through tilting. For example, with respect to a first axial half 701 of the actuator 70, the threading 75b can be along a circumferential portion of the inner diameter of the channel 79 that is on a first diametrical half/side S₁ of the actuator, whereas with respect to the other axial half 702 of the actuator 70, the threading 75a can be along a circumferential portion of the inner diameter of the channel 79 that is on an opposite diametrical half/side S₂ of the actuator relative to the half/side S₁.

Configuring the actuator 70 to the gross adjustment configuration (e.g., tilted configuration) can be desirable in various stages of an implantation procedure. For example, according to some practices, an initial cinching of the implant tension wire can be implemented in a manner such that a first amount of tensioning can be implemented, followed by a period of waiting to allow the cardiac cycling/motion to equalize the tension forces on the implant. Subsequent stages of tensioning can be implemented followed by appropriate wait times to avoid anchor detachment and/or other risks associated with tensioning the implant too quickly. In some cases, the amount of tensioning required to achieve the optimal tensioning of the tension wire may result in strain or fatigue for the operator. Therefore, it may be desirable to allow the operator to implement at least certain portions of the tensioning process by simply sliding the tension wire carriage 62, rather than rotating the actuator 70.

In some implementations, the operator is able to toggle between engaged and disengaged configurations of the actuator 70, as desired, to reduce the amount of knob rotation required of the operator when tensioning in the disengaged configuration.

In some implementations, such as the example of the actuator 70 shown and described above in connection with FIGS. 8A-8C, may allow for rapid uncinching/untensioning of the tension line, such that it is not necessary to rotate the actuator 70 to cinch the wire. That is, the tilting of the actuator 70 can function as a release mechanism, wherein the biasing of the tension line/wire, which can be based on the tension in the line and/or spring biasing implemented in connection with the tensioning device 60, causes the tension wire carriage 62 to slide distally when released from the threads 75 of the channel, thereby reducing the tension in the line.

In some implementations, the actuator 70 can be spring-biased, or otherwise biased in the engaged position/configuration, wherein the threaded channel 79 is aligned with and engaged with the wire tension carriage/bolt 62.

In some implementations, the operator may be required to hold the actuator 70 in the disengaged/tilted position continuously during gross tension adjustment, wherein release of the actuator 70 causes the actuator to reengage and/or tilt back to the engaged position/configuration (e.g., position aligned with the axis of the carriage 62).

In some implementations, the actuator 70 can be biased to the disengaged configuration/position. For example, the device 60 can include a mechanism to hold the knob 70 in the engaged position, wherein removal/release of such mechanism can allow the actuator 72 tilt, wherein spring biasing associated therewith can cause the actuator 70 to tilt to the disengaged configuration.

In some implementations, a button, slider, or other mechanism can be pressed, slid, or engaged to release the actuator from the engaged configuration, thereby allowing the operator to disengage the nut, or the nut can automatically assume the disengaged position/configuration when the button or other mechanism is engaged or released.

In some implementations, a locking mechanism can be included that allows the operator to lock the actuator in the disengaged when tilted or otherwise placed in the disengaged configuration.

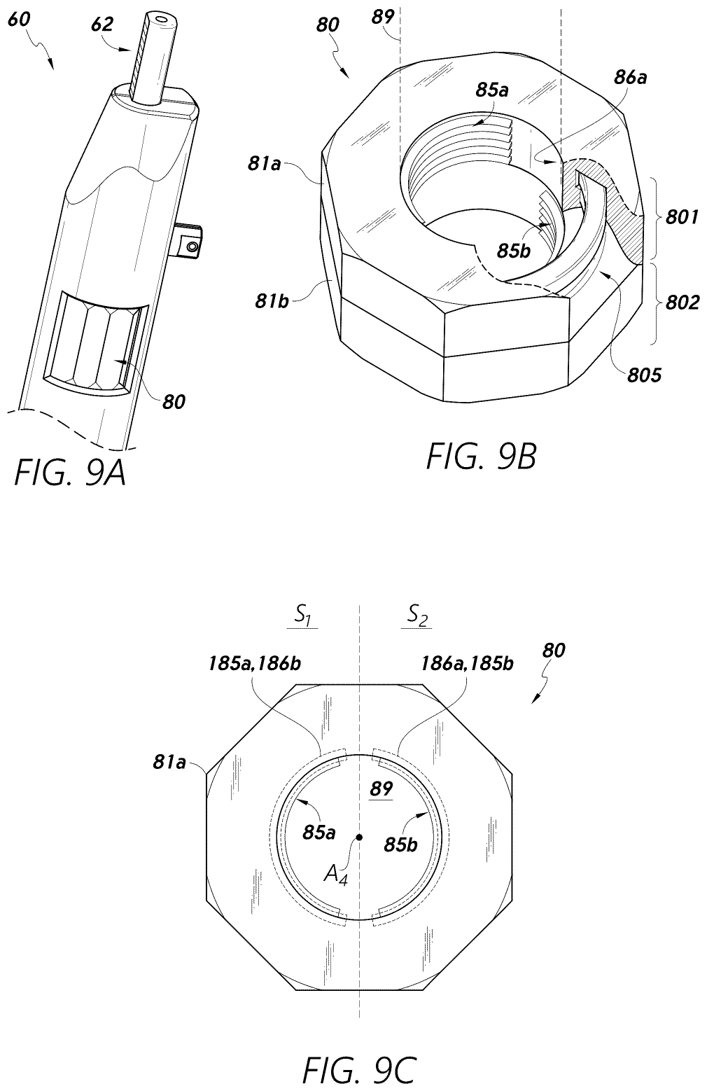

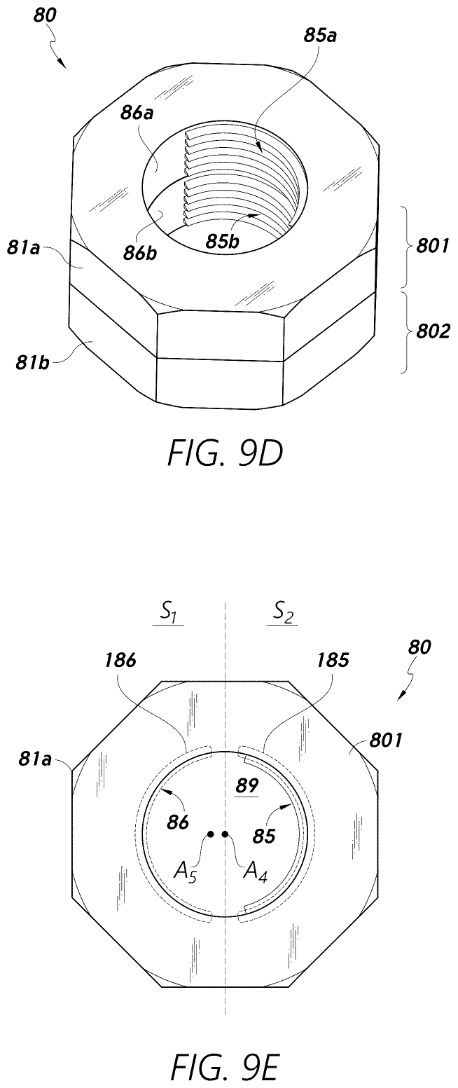

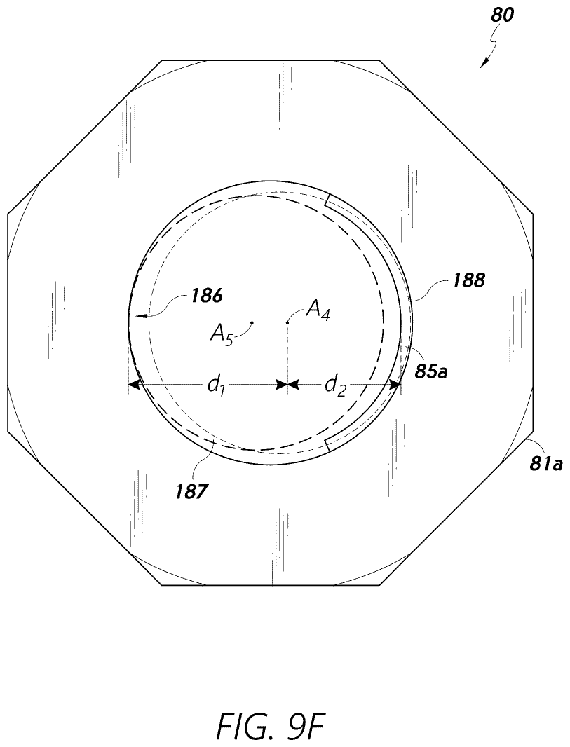

FIGS. 9B and 9C show perspective and axial views, respectively, of an example tensioning actuator 80 in an engaged configuration in accordance with one or more implementations. The tensioning actuator can be a component of a tensioning device 60 as shown in FIG. 9A, or any other tensioning device disclosed herein. FIGS. 9D and 9E show perspective and axial views, respectively, of the tensioning actuator 80 in a disengaged configuration in accordance with one or more implementations. FIG. 9F shows an axial view of engaged and disengaged tension wire carriage channels 187, 188 for the tensioning actuator 80 of FIGS. 9A-9E in accordance with one or more implementations.

Unlike the example actuator 70 of FIGS. 8A-8C, which is a tilt-based quick-release actuator, the example actuator 80 of FIGS. 9A-9E is a rotation-based quick-release actuator, wherein rotation of a first axial segment 801 relative to a second axial segment 802 can be implemented to selectively engage and/or disengage threads 85 associated with the respective axial segments 801, 802 with corresponding threads of the wire tension carriage/bolt 62. For example, each axial segment 801, 802 can have an inner diameter that defines a channel 89 through the actuator 80, wherein a first circumferential portion thereof 185 (see FIG. 9C) is associated with threading 85, whereas the remaining circumferential portion 186 is associated with relatively smooth surface 86.

In the engaged configuration, the threaded portion 185a of the first axial segment 801 can be positioned entirely or mostly on a first diametrical side S₁ of the actuator 80, whereas the threaded portion 185b of the second axial segment 802 can be positioned entirely or mostly on a second diametrical side S₂ (see FIG. 9A; the visible threading in the dashed outline 186a. 185b corresponds to the treaded portion 185b of the second axial segment 802, whereas no threads are associated with the surface 186 that is in front of the threading 185b with respect to the illustrated orientation).

In some implementations, the threads 85, which project inwardly from the inner diameter of the channel 89 can engage with the threads of the carriage/bolt 62 in a manner as to align the central axis A₄ of the actuator 80 with the axis of the bolt/carriage 62.

In some implementations, rotation of the first 801 and second 802 axial segments of the actuator 80 together can axially actuate the tension line carriage 62 relative to the actuator 80. In order to rotate the axial segments 801, 802 together, without rotation of the axial segments relative to one another, any suitable or desirable mechanism can be implemented to lock the axial segments rotationally relative to one another. For example, a cover, sleeve, peg, tab, flange, ridge, groove, or other structural feature or mechanism can be implemented to engage with the actuator 80 to hold the axial segments 801, 802 in a relative fixed rotational position. Such mechanism/means can be engaged or manipulated by the operator to transition between adjustable and non-adjustable configurations of the actuator 80, wherein in the adjustable configuration, the first axial segment 801 is rotatable relative to the second axial segment 802.

In some implementations, in order to release the line carriage 62 from the threads 85 of the actuator 80 to thereby allow for gross tensioning of the tension line, the first axial segment 801 can be rotated (e.g., manually by the operator) about a central axis A₁, relative to the second axial segment 802, such that the threaded portion 185a of the first axial segment 801 at least partially overlaps and/or aligns with the threaded portion 185b of the second axial segment 802. In some implementations, both threaded portions 185 and associated threads 85 can advantageously be entirely, or primarily, on a common diametrical side of the actuator 80. Such rotation can further at least partially align/overlap the non-threaded portions 186 of the axial segments, as shown in FIG. 9E. In some implementations, the tension line/wire carriage 62 can be permitted to disengage from the threads 85 and move axially relative to the actuator 80 (e.g., through movement of the actuator 80 or the carriage 62), such that the threads of the carriage 62 move away from the threads 85 towards the diametrical side associated primarily with the non-threaded portions 186 of the axial segments 801, 802. For example, spring or other biasing can be implemented to bias the relative position of the carriage 62 and actuator 80 toward the non-threaded/smooth side of the actuator 80. In some implementations, such biasing can be with respect to the second axial segment 802, wherein the biasing can be towards the smooth/non-threaded portion 186b of the second axial segment 802, which can likewise be towards the non-threaded portion 186a of the first axial segment 801 when the axial segments are configured in the disengaged configuration shown in FIGS. 9D and 9E.

In some implementations, the actuator 80 can include a rail/track feature 805, which can be configured to hold the first segment 801 and the second segment 802 together in a slidable engagement. In some implementations, the rail/track feature 805 can include a projecting rail portion associated with one of the axial segments 801, 802 and a recessed track feature configured to receive and/or slide over the rail, such feature being associated with the other of the segments 801, 802.

In some implementations, when the actuator 80 is in the disengaged configuration of FIGS. 9D and 9E, the axis A₁ of the tension line/wire carriage 62 can be offset from the center axis A₄ of the actuator 80 due to the biasing toward one side of the actuator. That is, whereas in the thread-engaged configuration, the bolt/carriage can occupy a space 188 overlapping the threads 85 and co-axially positioned with the central axis A₄, in the disengaged configuration, the bolt can occupy an offset area 187 (see FIG. 9F) that is defined by and/or adjacent to the non-threaded portion 186 of the actuator. For example, with respect to the central axis A₄ of the actuator 80, the distance d₁ between the axis A₄ and the non-threaded wall 186 can be greater than the distance d₂ between the axis A₄ and the inner-most extension of the threads 85 in the threaded portion 185. Such greater distance d₁ can allow for the diameter/area of the bolt 62 to fit in the offset area 187 without engaging with the threads 85. Therefore, in the disengaged configuration of FIGS. 9D and 9E, the tension line carriage/bolt 62 can be permitted to slide axially in the offset area 187, away from the threads 85, wherein the carriage/bolt 62 can be held in the area 187 by a biasing means/mechanism (e.g., spring(s)).

In some implementations, with the subunits/segments 801, 802 rotated 180° from the engaged configuration of FIGS. 9B, 9C, the open/smooth parts/walls 186 of the subunits/segments are aligned, and the threaded parts 185 can be moved away from the threaded bolt 62. The biasing means/mechanism can be utilized to push the actuator 80 laterally away from the bolt 62.

In some implementations, the rotatable knob portion of the actuator 80 can be a separate component from the rotatable segments 801, 802, wherein the knob component can be placed over the nut components/segments 801, 802 to allow for common rotation of both segments.

In some implementations, the knob can be axially slid away from the nut components 801, 802, such as by using some type of cam surface, gear or meshing, or other mechanical coupling/engagement surface/feature between the knob component and the nut components 801, 802, such that when the knob component is moved away or disengage from the nut components 801, 802, the nut components can be accessible for rotation thereof. Re-engagement of the knob component with the nut components 801, 802 can force the nut components 801, 802 into a relatively locked configuration, thereby allowing for rotation of the nut components together such that they can be rotated together as one unit.

The examples of FIGS. 8 and 9 allow for a device that can be actuated by a knob to be released relatively quickly and easily without introducing undue risk to the procedure. Such quick-release gross-tensioning may typically not be implemented during cinching of the implant device. That is, the gross-tensioning described above may often/generally not be implemented to increase tension in the cinching wire, but rather to release tension relatively quickly.

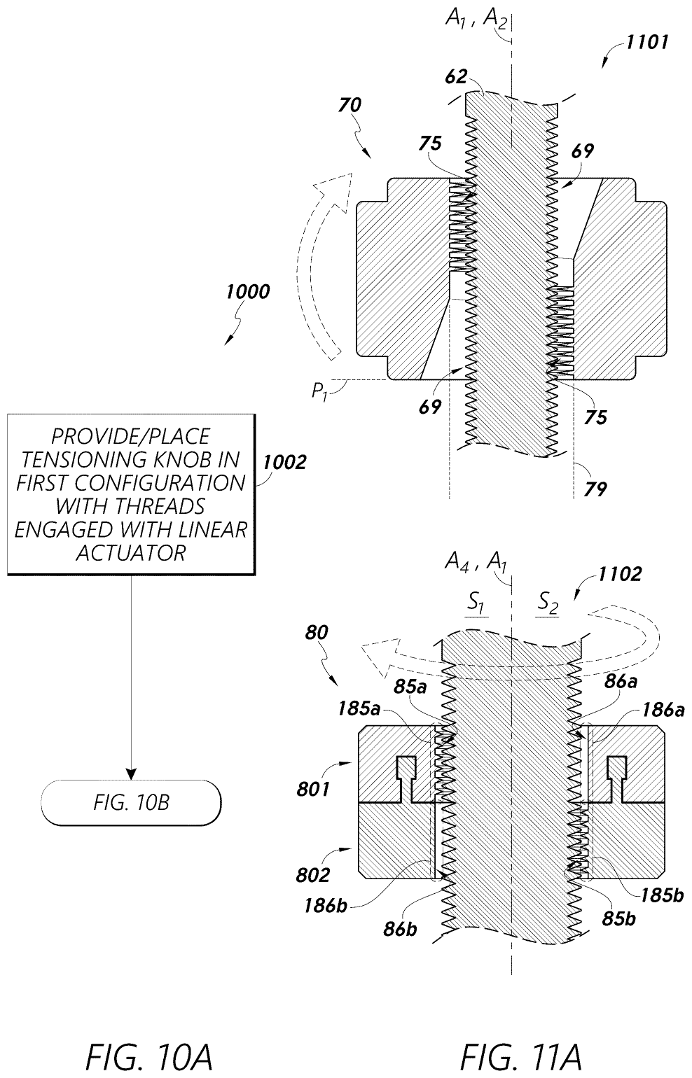

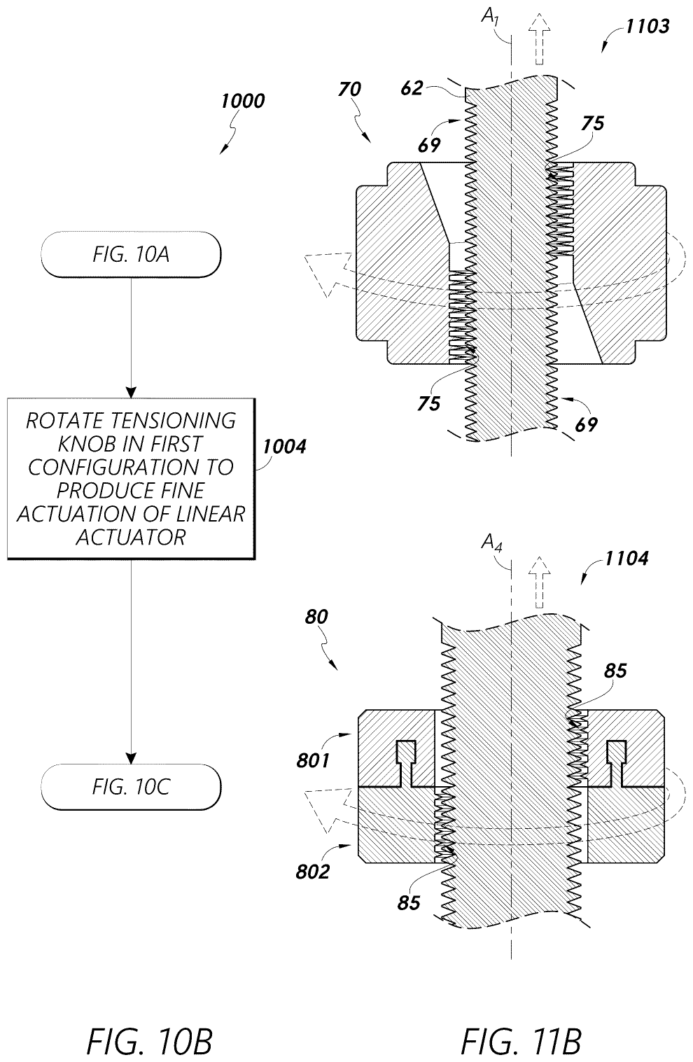

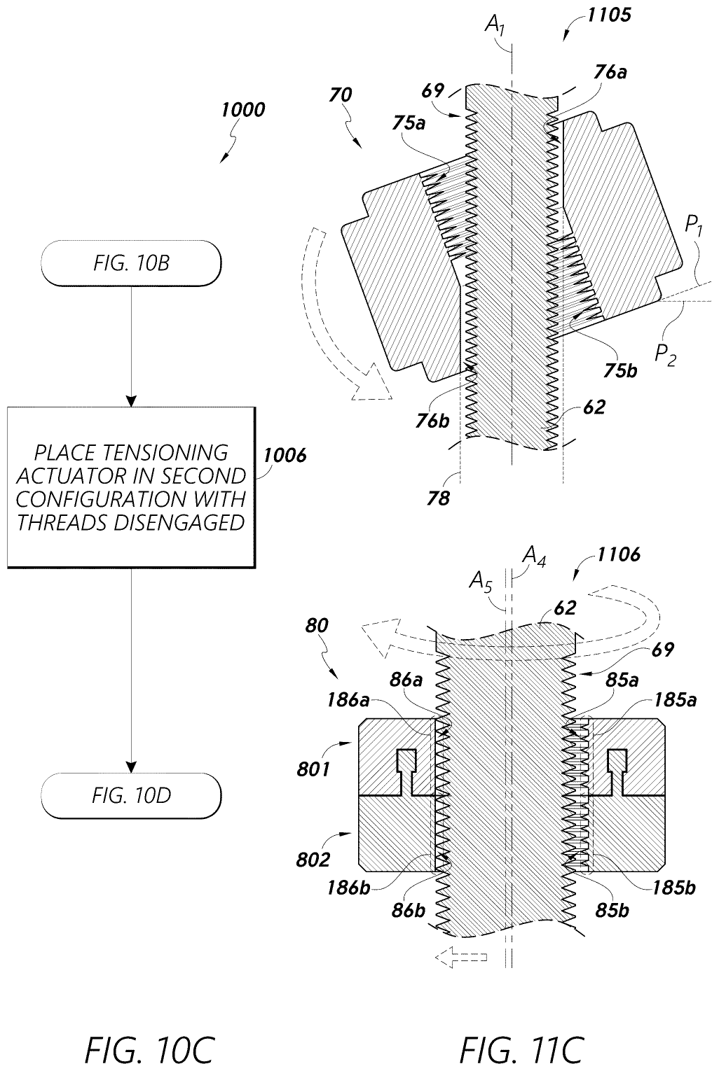

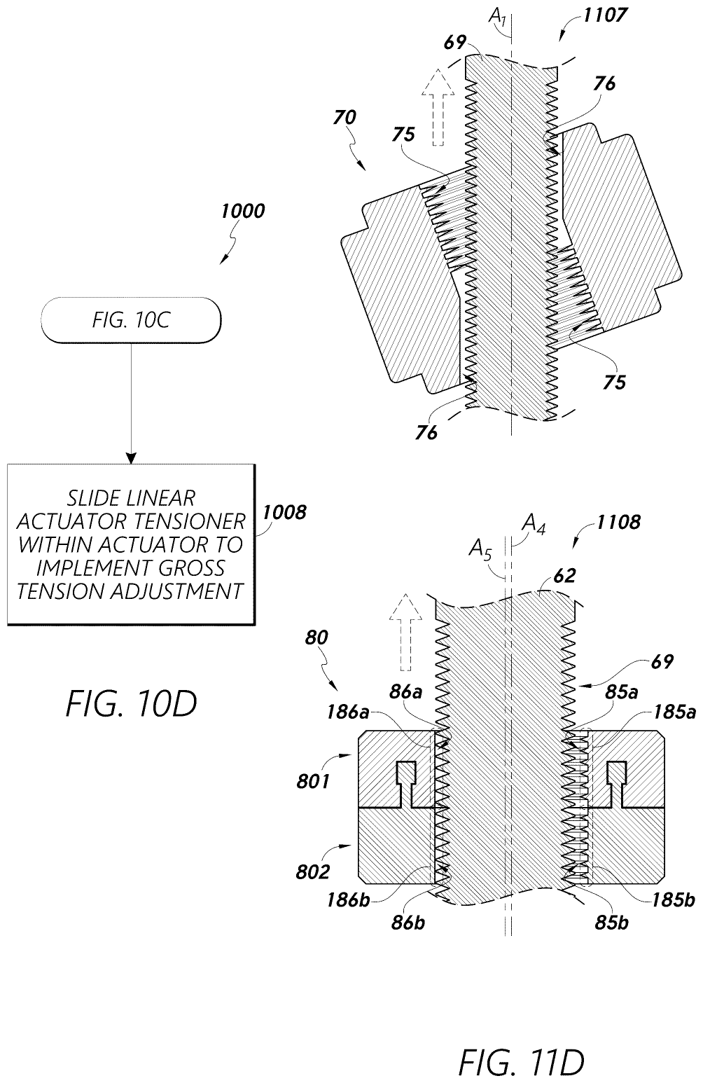

FIGS. 10A, 10B, 10C, and 10D provide a flow diagram illustrating an example process 1000 for tensioning a tension line/wire associated with an implant device in accordance with one or more implementations. For example, the process 1000 can be implemented to perform gross and fine tensioning/actuation with respect to a linear actuator tensioner. FIGS. 11A, 11B, 11C, and 11D provide images of certain devices/components corresponding to operations of the process 1000 of FIGS. 10A, 10B, 10C, and 10D in accordance with some implementations. The process 1000 can be implemented, for example, to repair a dilated annulus of a patient and/or to treat a cardiac valve of the patient (e.g., aortic valve, pulmonary valve, mitral valve, tricuspid valve), although it should be understood that the process 1000 can be implemented in connection with operations on other anatomy, or even in non-medical settings for tensioning lines/wires in other implementations. The process 1000 can be implemented following anchoring of an implant device to tissue of, for example, a native heart valve annulus, which can be performed using any suitable or desirable type of anchoring devices/implant.

At block 1002, the process 1000 can involve providing and/or placing a tensioning actuator, such as a manually-manipulatable actuator associated with a handle of a tensioning device, in a first configuration in which threads of the actuator are engaged with corresponding threads of a tension line carriage/bolt. Such configuration can be considered and engaged configuration, as described in connection with various implementations disclosed herein.

Images 1101 and 1102 show example implementations of actuators 70, 80 that can be used in connection with the process 1000. For example, image 1101 shows a tilt-based quick-release actuator 70 in an engaged configuration in which inner threads 75 associated with a first pseudo-channel 79 of the actuator 70 are engaged with the threads 69 of the tension wire carriage/bolt 62.

In accordance with some implementations, image 1102 shows a rotation-based quick-release actuator 80 in an engaged configuration in which axial segments/units 801, 802 of the actuator 80 are relatively rotationally positioned in a manner such that threaded portions 185 of each segment/unit 801, 802 are on opposite diametrical sides S₁/S₂ and/or at least partially non-overlapping, such that the threads 85 are engaged with the threads 69 of the bolt/carriage 62 on opposite diametrical sides thereof and hold the bolt/carriage 62 in alignment with the central axis A₄ of the actuator 80.

At block 1004, the process 1000 can involve rotating the relevant actuator in the engaged configuration to produce fine linear actuation of the tension line carriage/bolt 62. That is, the threads of the actuator and/or carriage can be designed such that rotation of the actuator produces minimal and/or relatively fine linear movement in the carriage/bolt 62.

Image 1103 shows the example tilt-based/configurable quick-release actuator 70 in the engaged configuration shown in image 1101, wherein the actuator 70 is rotated about the axis A₁ of the bolt 62 to produce axial movement/translation in the bolt/carriage 62. Image 1104 shows the example rotation-based/configurable quick-release actuator 80 in the engaged configuration in which threads 85 of the segments 801, 802 are at least partially non-overlapping, as shown in image 1102, wherein the actuator 80 is shown being rotated to produce relatively fine axial movement/translation of the carriage/bolt 62.

At block 1006, the process 1000 can involve placing the actuator in a second configuration in which the threads thereof are disengage from the threads 69 of the tension line bolt/carriage 62. That is, the actuator can be placed in a disengaged, released configuration in connection with the operation a block 1006. Image 1105 shows the tilt-based quick-release actuator 70 in a second configuration, such as a tilted configuration with respect to the axis Aⱼ of the carriage/bolt 62, wherein the threaded portions 75 of the inner diameter of the actuator 70 are disengaged from the threads 69 of the bolt/carriage 62, and the non-threaded walls 76 are moved into alignment and/or contact with the tension line/wire carriage 62.

In image 1106, an implementation of the actuator 80, which is in the form of a rotation-based quick-release actuator 80, is shown in a disengaged configuration in which the threaded portions 185 of the inner diameter of the actuator 80 are shown as at least partially overlapping with respect to the circumference/arc thereof, which can be achieved by rotating the first segment 801 and second segment 802 relative to one another, which can be performed manually in some implementations. Alignment of the threaded and/or non-threaded inner wall portions of the actuator 80 may cause or allow the bolt 62 to move laterally/transversely away from the threaded wall portions 185 towards the non-threaded wall portions 186 to thereby release/disengage from the threads 85.

At block 1008, the process 1000 can involve sliding the tension wire carriage 62 disengaged from the actuator threads in a manner as to produce gross/free tensioning movement of the carriage 62. For example, such sliding may be achieved through manually pushing or pulling the carriage 62, or at least partially through automatic biased movement of the carriage, which may be caused by certain biasing mechanism(s)/mean(s) (e.g., spring(s)). Sliding of the tension line/wire carriage 62 is shown in images 1107, 1108.

Electronic and/or Motorized Tensioning and Tension Sensing

As described in detail above, certain medical procedures can involve the cinching of implant coupling wires/lines after and/or during deployment of an implant device/assembly. Such tensioning can be implemented using a tensioning device, which can be embodied at least partially in a handle-type device/assembly. While certain implementations are described above relating to manual tensioning of tension lines/wires through engagement with manual linear actuator components, manual rotation or other engagement of/with actuator components can cause or involve undesired strain, attention/focus, and/or time, as described above. Some implementations of the present disclosure provide improved ease-of-use, precision, and/or speed with respect to tension wire/line carriage actuation through the use of electronic and/or motorized components. Such implementations can further allow for tension monitoring using various electronic and/or motorized mechanisms. For example, monitoring absolute and/or relative tension levels associated with an implant line/tether can improve outcomes and/or reduce risk of injury or damage to the patient and/or instrumentation.

In some implementations, electronically-controlled and/or motorized actuator assemblies can be implemented within a tensioning device/handle as a means/mechanism to drive the tensioning carriage for the purpose of tensioning an implant-tensioning line. Electronic monitoring and/or limiting of the applied tension is also disclosed herein in connection with some implementations presented below, although it should be understood that tension-monitoring concepts described below can be implemented in connection with any implementation disclosed herein. Implementations of the present disclosure can provide real-time measurements of cinching forces, thereby allowing the operator/surgeon to make decisions and/or take actions based on present known tension conditions.

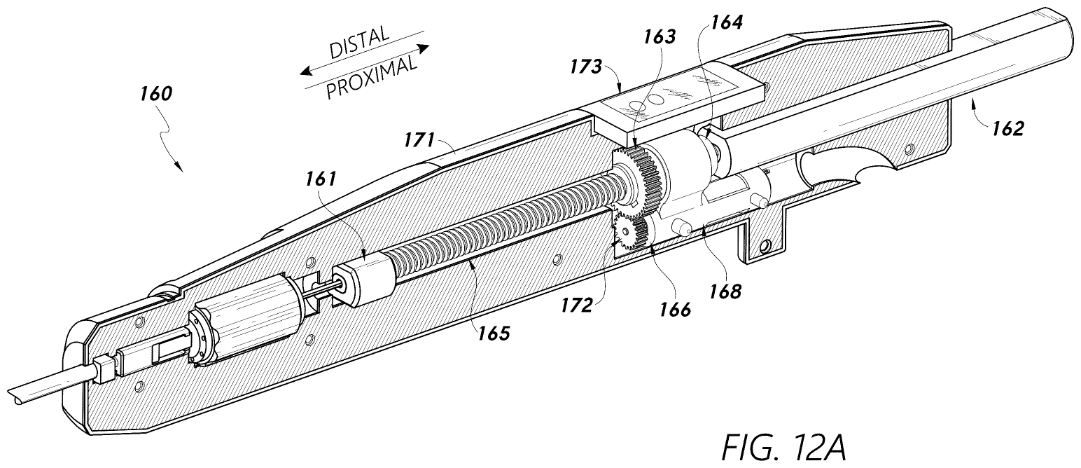

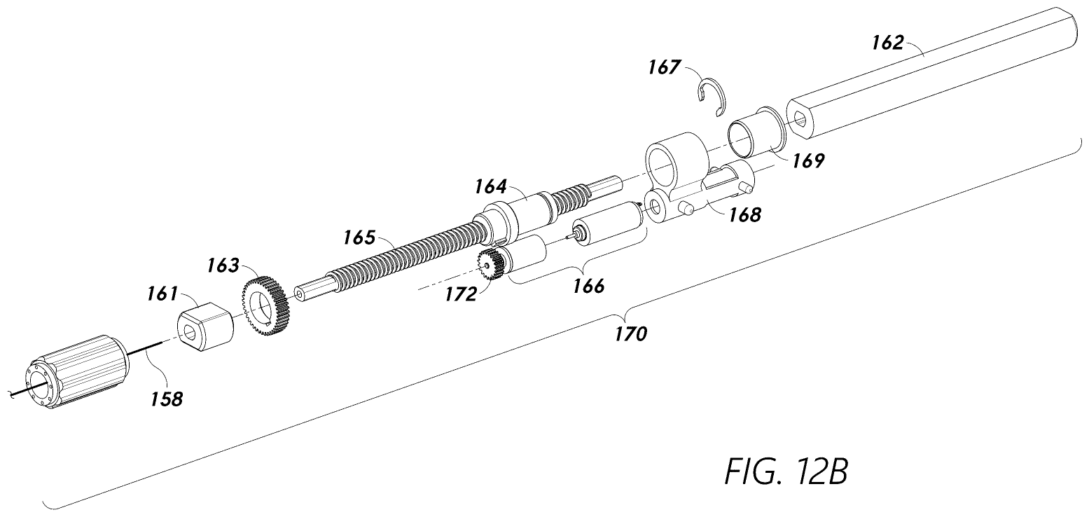

FIGS. 12A and 12B show cutaway and exploded views, respectively, of components of an example motorized tensioning device 160 in accordance with one or more implementations. Motorized actuation of the tensioning assembly 170 of the tensioning device 160 can be facilitated by an electronic motor 166 and can advantageously provide for continuous, controlled tensioning/cinching of a tensioning line 158. Furthermore, the motor 166 can be controlled in a manner as to define the tensioning rate to a desirable value. In some implementations, real-time tension line/wire force measurements may be derived based at least in part on motor parameters and/or other means or mechanism(s) for measuring tension in the tensioning wire.

In some implementations, the tensioning device 160 includes a handle housing 171, which can house certain tensioning components, as with the manual tensioning implementations described above. In some implementations, the electrical motor 166 can be provided within and/or otherwise associated with the housing 171. In some implementations, the tensioning device 160 can comprise a user input/control interface 173, which can comprise one or more buttons or other mechanisms for providing input for controlling the motor 166. Button-based control of the electric motor 166 can obviate the need for the operator to manually turn a knob or other actuator as a means of adjusting tension in the line/wire 158.

In some implementations, the motor 166 can have associated therewith a gear 172, which can be meshed with a gear 163 that is rotatably fixed with a rotational-to-axial motion translation structure 164, which can be in threaded engagement with a rod 165 that is coupled to and/or otherwise associated with the tension line carriage 162. In some implementations, the tension line carriage 162, as with other implementations disclosed herein, is fixedly attached to the tension wire 158 at/in some area thereof. For example, the wire/line 158 can be fixed to the carriage 162 at or near a proximal portion thereof, or any other portion or area of the carriage.

In some implementations, the motorized cinching mechanisms shown in FIGS. 12A and 12B can provide safety features that can improve patient outcomes. For example, electronic control can be implemented to control the amount of tension on the control wire 158 and maintain tension within a desirable range or at a desirable level. Such tension levels can be customized to the particular patient in some cases. In some implementations, safety thresholds can be set based on line tension measurements, thereby reducing the risk of injury or damage.

In some implementations, the wire 158 can run through some of the components of the motorized tension actuator assembly 170. For example, the wire 158 can be passed through at least a linear adapter 161 component, translation rod 165 and tension wire carriage 162. In some implementations, the carriage 162 has one or more flat surfaces, wherein such surface(s) can facilitate rotational locking of the carriage 162 to prevent rotation thereof when the rotational-to-linear translation structure 164 is rotated, thereby inducing linear/axial translation of the rod 165.

In some implementations, the axial translation rod 165 likewise can be restrained from rotational movement for the same purpose. For example, such component can be mechanically coupled to the carriage 162 and/or a distal adapter 161, which can likewise have one or more flat surfaces designed to provide for rotational restraint. In some implementations, the rotational-to-linear translation structure 164 can comprise a plastic bearing or similar component/structure, which can have internal threading that meshes with the threading of the translation rod 165 to thereby cause actuation of the rod as the structure 164 rotates thereabout.

In some implementations, the translation structure 164 can have certain flanges and/or other interference structural features, which can serve to retain the various components in the desired positions and orientations within the assembly 170.

In some implementations, the assembly 170 can comprise a bushing or bearing feature 169, which can facilitate smooth rotation of the translation structure 164 within a motor housing structure 168. In some implementations, the bushing/bearing 169 can be press-fitted into the housing 168 and can create a relatively smooth interface between the rotating translation structure 164 and the static housing structure 168. In some implementations, a C-clip or similar structure 167 can be used to prevent axial movement of the bushing/bearing 169 relative to the translation structure 164 by nesting within a groove of the structure 164 and providing radially-projecting obstruction.

In some implementations, a proximal end of the translation rod 165 can mate with the tension wire carriage 162 in a distal receptacle thereof. Such mating can be via a press-fitting engagement. It may be advantageous for the rod 165 to be fixedly attached to the carriage 162, such that any axial movement/translation of the rod 165 is translated to the carriage 162.

In some implementations, the interface 173 can be implemented to provide output to the user that indicates tension levels of the tension wire/line. For example, such output to the operator may be visual, audible, tactile, or in any other form. In some implementations, tension level output may be provided as a constant readout on the interface 173. In some implementations, output may be provided only in periods of high (or low) tension levels as a mechanism to warn the user of tension conditions and associated risks.

In some implementations, the interface 173 can comprise one or more buttons which can be engaged to activate the motor in the tension increasing or reducing directions. In some implementations, the button can be pressed to tighten the line 158 (i.e., increase tension), whereas in other implementations, the button can be pressed to loosen the line (i.e., decrease tension). In some implementations, the interface 173 can comprise one or more buttons for tightening as well as one or more buttons for loosening the tension in the line/wire 158. In some implementations, the button(s) can be continuously pressed in order to activate the motor, wherein release of the button(s) causes the motor to stop. Alternatively, the pressing and releasing of the button(s) can turn on the motor, whereas subsequent pressing and releasing of one or more buttons can be required to cease operation thereof.

In some implementations, the motor 166 can be configured to translate rotation to the gear 163 in a manner as to implement specific speeds of linear actuation of the rod 165. For example, in some implementations, the motor 166 can be configured to actuate the rod 165 to produce a linearly constant speed of at least 2 mm/s. The various threads implemented for the purpose of linear actuation can comprise rectangular threads in some implementations, which can be advantageously efficient relative to alternative implementations. The threads can have an optimal pitch angle, such as about 30° (e.g., 29°), or other value.

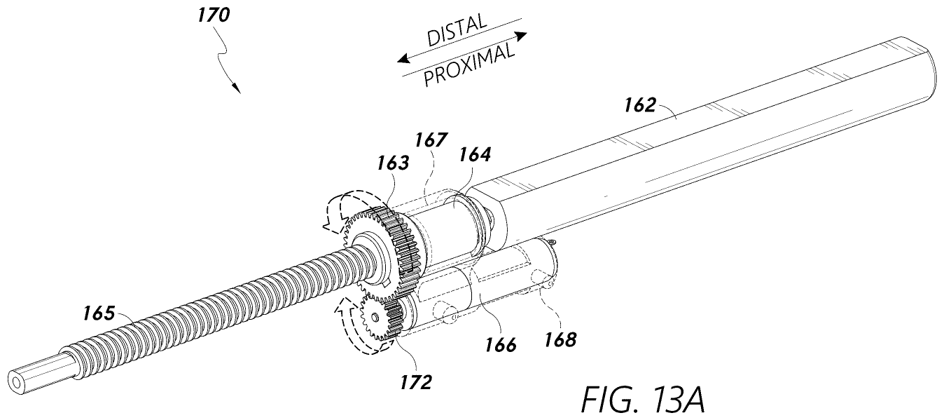

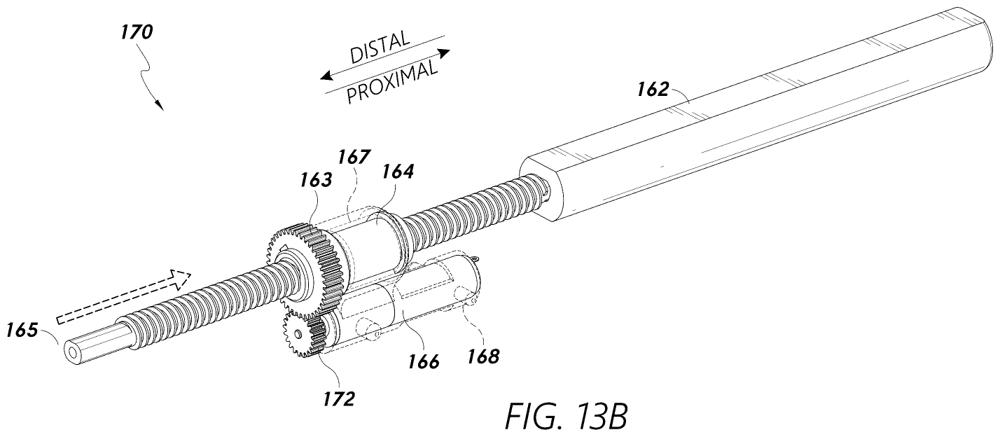

FIGS. 13A and 13B show components of a motorized tensioning assembly in various actuation states in accordance with one or more implementations. Specifically, FIG. 13A shows a more distal position of the line carriage 162 and translation rod 165, corresponding to a relatively slack line/wire, whereas FIG. 13B shows a more proximal position of the line carriage 162 and translation rod 165 after some amount of motorized actuation of the gear 163 and translation rod 164, corresponding to a relatively higher tension in the tension line.

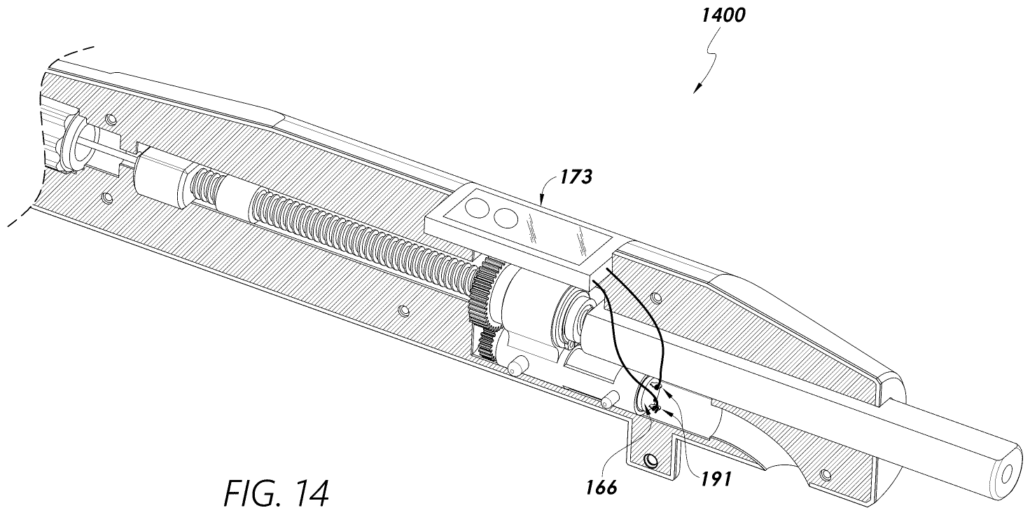

As referenced above, use of electrical and/or motorized tensioning actuation may allow for the measuring of implant wire forces in some manner. For example, in some implementations, line/wire tension may be measured based at least in part on electrical motor resistance. FIG. 14 shows a tensioning device 1400 with motor-resistance-based tension reading functionality in accordance with one or more implementations.

In some implementations, electronic monitoring of applied tension to the tension wire/line can be achieved using motor resistance as a surrogate for wire tension. For example, for a given voltage, higher levels of mechanical resistance can result in relatively lower current due to mechanical resistance on the motor, which causes electrical resistance within the motor. With respect to driving the motor 166, mechanical resistance (e.g., due to tension in the line that is indirectly coupled to the translation rod 165 driven by the motor 166) can be compensated for by increasing voltage levels applied to drive the motor, which may be necessary or desirable to maintain a constant speed of movement of the carriage 162. Such levels can be detectable in the form of detected motor resistance by electrically coupling with the electrical motor 166 in a manner as to measure the resistance of the motor.

In some implementations, electrical contacts 191, which can have opposite polarity in some instances, can contact the motor 166 in a manner as to provide signal(s) indicative of electrical resistance of the motor. For example, the electrical contacts 191 can provide a mechanism of converting resistance of the motor to tension readout on the interface 173, which can inform the operator as to tension conditions with respect to the implant tether/wire.

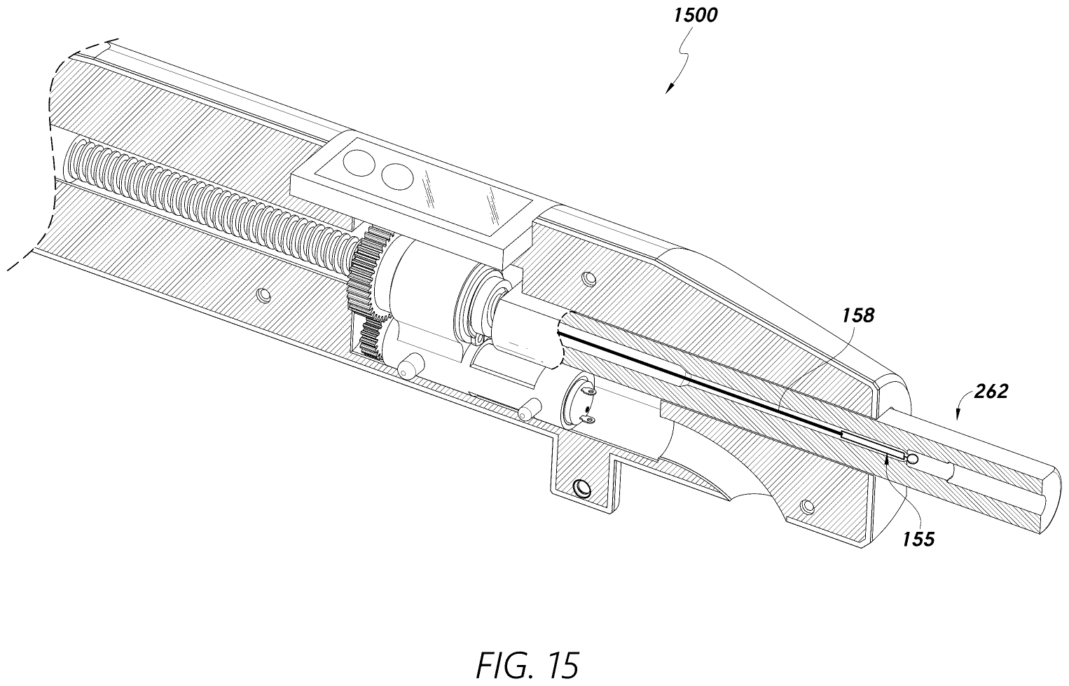

In addition to determining wire tension based on electrical motor resistance, implementations of the present disclosure can provide tension-determining functionality based on strain gauge sensors/devices and/or other mechanisms. FIG. 15 shows a tensioning device 1500 with strain-gauge-based tension reading functionality in accordance with one or more implementations. In some implementations, an in-line strain gauge 155 can be coupled between the tension wire carriage 262 and the tension wire 258 itself.

In some implementations, the strain gauge 155 can be electrically coupled to the interface 173 and/or control circuitry associated therewith, wherein readings from the strain gauge 155 can be used to determine/derive the present tension levels in the line 158. The strain gauge 155 can be coupled to the component(s) of the device 1500 in any manner, wherein the strain on the strain gauge 155 indicates tension in the wire 258. For example, as referenced above, the wire 258 can be mechanically/physically coupled (directly or indirectly) to the strain gauge on/at one end thereof, whereas an opposite end or portion of the strain gauge can be mechanically/physically coupled (directly or indirectly) to the carriage 262. That is, the wire 258 can be coupled to the carriage 262 via the strain gauge 55. In some implementations, the strain gauge 55 is connected between the tension wire carriage 262 and/or other structural component of the handle 1500.

Determined tension levels may inform the operator and/or the device/system regarding tensioning adjustments to be made. For example, in some implementations, tension devices include certain control circuitry configured to alter the tension, through linear actuation, in the implant tether/wire in response to determined tension levels. For example, the control circuitry may be configured to adjust the rate of rotation of the motor gears to increase or decrease the tensioning (e.g., tightening or loosening) of the tension wire in response to the determined tension level reaching a predetermined threshold or other condition. For example, tension may be determined based on electrical motor resistance and/or strain gauge readings, wherein control circuitry of the tensioning device/system is configured to implement responsive tension adjustment based on such readings.

Tensioning Line with Signal Filaments

As described in detail above, some implementations of the present disclosure relate to implant devices/assemblies configured to cinch target tissue, such as a heart valve annulus, to a smaller size. Some implementations are configured to achieve such cinching at least in part by deploying anchors into the target tissue and cinching the anchors closer together using a tensioning line (e.g., cable, wire, tether, suture, etc.) that is coupled to each of the anchors. For example, the tensioning line can be threaded through coupling means of anchor heads of the tissue anchors. In order to reduce friction between the tensioning line and the line-coupling means of the anchors (e.g., eyelet, hook, or the like), the particular geometric tolerances and materials implemented for the tensioning line can be selected to provide the desired characteristics. However, friction between the tensioning line and the anchor coupling means (e.g., eyelet), as well as potential kinking of the tensioning line can contribute to degeneration of the tensile strength of the tensioning line and/or prevent free sliding of the tensioning line through the anchor coupling means during deployment.

As described above, it can be desirable to determine present tension force conditions of an implant tensioning line in order to prevent or reduce the risk of tension line breakage or damage, which can result in injury to the patient and/or other complications. Implementations of the present disclosure provide various means for determining tension in an implant tensioning line, some of which are disclosed above. In addition to the concepts disclosed above, tension determination according to aspects of the present disclosure can involve implementing implant tensioning lines that integrate filaments that are configured to provide tension-indicating signals and/or features. Such filament integration advantageously can be implemented in a tensioning line construction that maintains and/or provides suitable or desirable break-load, profile, friction, and/or wire stiffness characteristics. Furthermore, such implementations can advantageously include certain echogenic/radiopacity features that enhance the visibility of the tensioning lines under imaging according to various modalities.

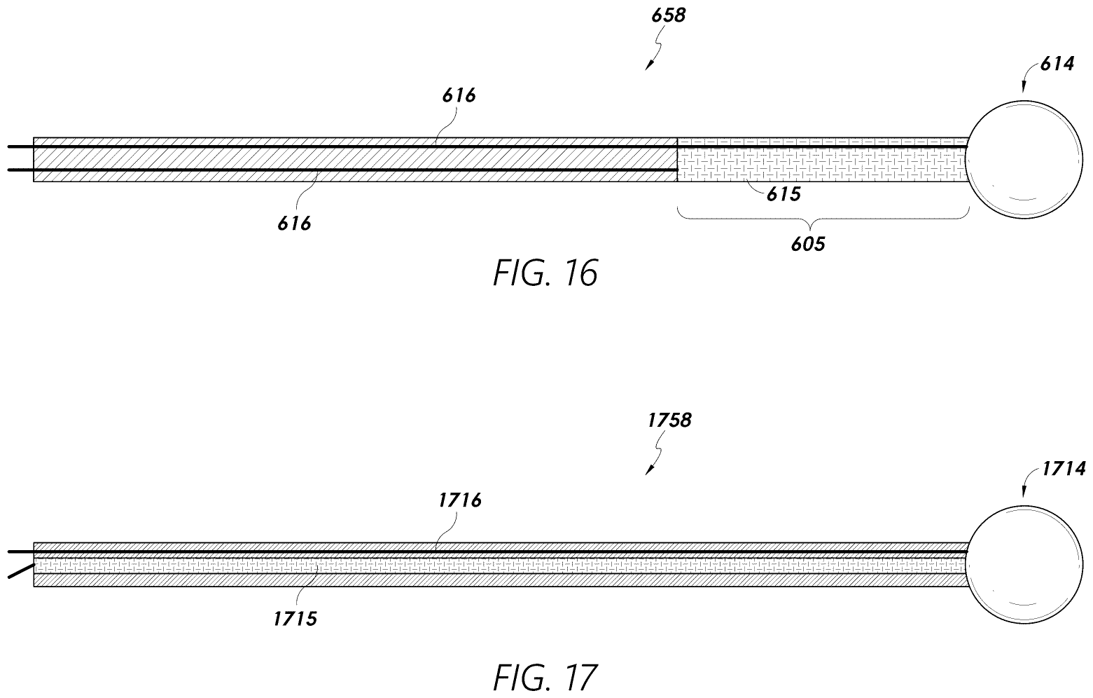

FIG. 16 shows a segment of an example tensioning line 658 including integrated tension-indicating features in accordance with one or more implementations. The tensioning line 658 includes a segment thereof 605 that includes one or more filaments or other elements 615 that have variable resistance that is dependent at least in part on stretch conditions thereof. For example, the stretch-indicating element(s) 615 can comprise one or more filaments that have an electrical resistance that changes based on the amount of stretch experienced thereby. In some implementations, the element(s) 615 can be used to gauge, monitor, and/or control tension forces on the line 658 during tensioning/cinching.

In some implementations, the strain-indicating element(s) 615 can comprise electrically-conductive material, wherein electrical current can be injected and/or flow through the conductive material (e.g., gold) to provide information relating to the resistance of the element(s). For example, a voltage drop across the element(s) 615 can be determined, wherein the resistance of the element(s) 615 can be derived based at least in part on the voltage drop. With such construction, the line 658 can act as a strain-gauge-type wire/line through incorporation of the filament(s)/element(s) 615 that has/have variable resistance based on elongation thereof; the force in the line 658 can be calculated based on the resistance in the signal filament(s)/element(s).

In some implementations, the variable-resistance, strain-indicating conductor filament(s)/element(s) 616 can form part of a closed electrical circuit that allows for electrical current to flow through the element(s). In some implementations, the stretch filament(s)/element(s) 615 can be electrically coupled to a printed circuit board and/or other circuitry (e.g., substrates, conductors, filters, amplifiers, and/or other processing elements) for signal transmission and/or processing.

In some implementations, such circuitry may be implemented in a terminal stopper 614, which may be in the form of a ball crimp and may be implemented at a terminal end of the line 658, as shown. In some implementations, wireless transmitter circuitry may be contained within the terminal stopper 614 to allow for post-operative strain monitoring.

Where multiple variable-resistance filaments are implemented, strain determinations may be based on a greatest determined strain among the plurality of conductors. In some implementations, strain determinations for the line may be based on an average strain of the conductors. Any suitable materials may be implemented for the conductive element of the variable-resistance filaments, such as various metals, including copper, nickel, iron, aluminum, platinum, gold, chromium, and metal alloys, including copper nickel (Cu—Ni), nickel chromium (Ni—Cr), nickel chromium modified alloys, nickel copper alloys, iron base alloys, platinum base alloys, gold base alloys, and the like. It may be necessary in some implementations for the material selected to be implantable grade, such as gold.

In the example shown in FIG. 16, the variable-resistance element(s) can be disposed primarily in, and/or limited to, a discrete section 605 of the line 658, such as a distal segment of the line. The section 605 may be considered an area of interest of the tensioning line. For example, the section 605 can be a distal portion of the line that is associated with the implant anchors. That is, the section 605 can be associated with the portion of the line 658 that is left behind as part of the implant after tensioning and delivery system removal. In some implementations, the segment of interest 605 is between 100-160 mm, such as about 140 mm. Such area 605 may be of particular concern due to the interaction with the anchor-coupling elements (e.g., eyelets) and due to the prolonged utilization of such portion of the line 658 as a permanent or semi-permanent implant.

In addition to, or as an alternative to, the variable-resistance filament(s)/element(s) 615, the tensioning line 658 can further include one or more additional signal filaments, which can be used for the purpose of transmitting data through the line 658. For example, various types of small-diameter filaments can be implemented through which electrical, optical, thermal, and/or mechanical signals can be transmitted through the line 658, such as to a proximal end thereof. Examples can include fiberoptic filaments, plastic filaments, metal filaments, and the like. In some implementations, such signal filaments can be interfaced with the variable-resistance stretch filament(s)/element(s) to forward/relay strain signals therefrom. In some implementations, optical signal filament(s) can be implemented to transmit light received at the distal segment 605 of the line 658, wherein such light data can be used to derive/determine regurgitant flow conditions at the target valve or other conditions associated with the target anatomy.

In some implementations, strain-indicating filaments can be implemented as mechanical binary strain indicators. For example, the filament(s) can be designed such that at a threshold strain, breakage occurs, thereby breaking an electrical circuit running through the filament. Monitoring of the strain of the line can involve monitoring whether a signal is present on the filament(s), wherein signal presence can be interpreted as the line being within suitable strain levels, whereas no signal can be interpreted as the strain of the line reaching, or exceeding, a maximum strain threshold. For example, in some implementations, cinching can be performed until the breakage of the strain-indicating filament(s), at which point further cinching may be ceased. Such filaments may be linked mechanically to some structure of the line 658, such as to the terminating stopper 614.

In some implementations, the filament(s) can be designed to break at a break force that is less than a maximum strain force for the line. The loss of signal on the line can provide a binary, switch-type reading indicating the tension in the line 658.

Any of the variable-resistance and/or other signal filaments integrated with tensioning lines disclosed herein can have electrical insulation associated therewith. For example, insulation may be necessary or desirable to prevent corruption of signals from electrical shunting/dissipation through fluid/blood or through other filaments of the tensioning line and/or tissue anchors that can result from non-insulated contact therewith. Furthermore, the implant may be disposed in the area near the atrioventricular (AV) node, and therefor without electrical insulation, the heart's electrical system may interfere with signal readings, and vice-versa, which could cause harm to the patient. Therefore, at least some of the filaments of the line 858 can comprise an electrically-insulating sheath or coating over at least a portion of the length thereof.

FIG. 17 shows an example of a strain-indicating tensioning line in which the variable-resistance filament(s)/element(s) 1715 is/are not limited to a segment of interest, but rather run substantially the entire length of the line 1758. In some implementations, the strain readings derived may represent an average strain of the entire line 658 rather that the strain of a sub-segment thereof. Therefore, where full-length variable-resistance filament(s) are implemented, it may be necessary or desirable to rely on relatively conservative strain thresholds, thereby potentially limiting the ability to cinch the line 1758, to avoid rupturing or over-straining the line 1758. For example, it may be assumed that at some points along the length of the line, the strain may be greater than at others. Therefore, where the strain concern is a weakest-link concern, the average strain may be lower than the assumed highest strain experienced in the line, requiring caution in cinching.

In some implementations, the line 1758 can include various signal filament(s)/element(s) 1716, as well as a terminal stopper 714.

The tensioning line 1758 and associated components can have any of the features described above in connection with FIG. 16.

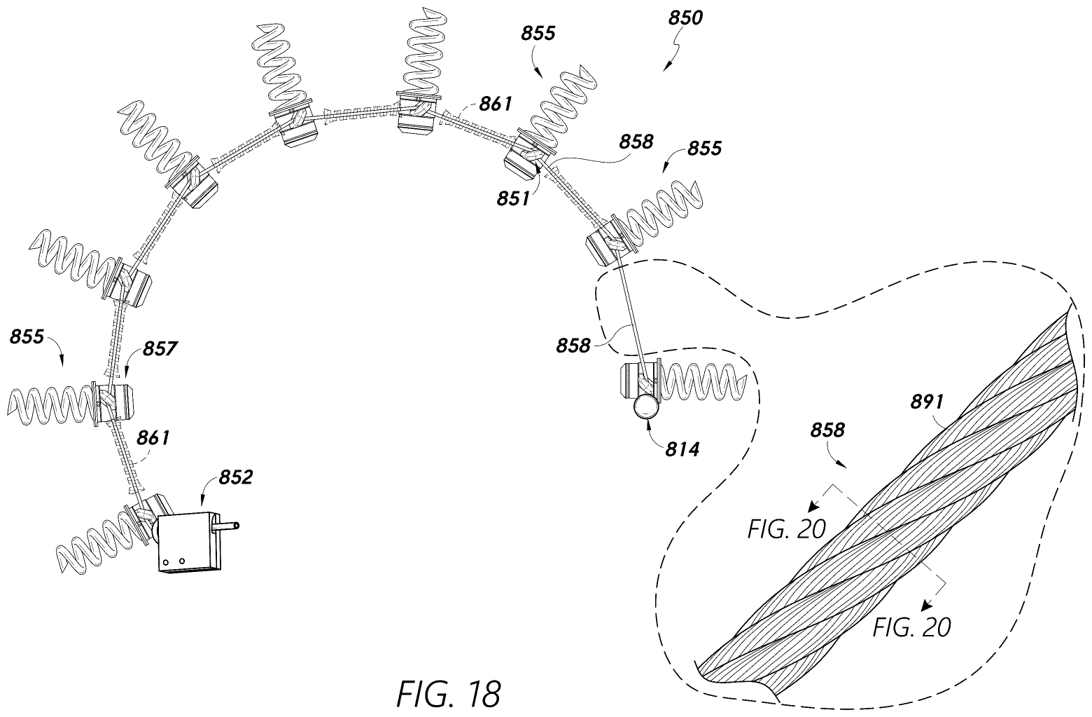

Tensioning lines in accordance with implementations of the present disclosure, including tensioning lines that include one or more variable-resistance strain filaments and/or other signal filaments, can have a cable design comprising multiple filaments (e.g., wires) wound together into strands, wherein the tensioning line may comprise multiple strands wound or braided together in some implementation. FIG. 18 shows an implant device 850 including a cable-type tensioning line 858.

When implanted with an annuloplasty or similar implant device/assembly, as shown in FIG. 18, the tensioning line 858 may generally follow a curved path (e.g., a semicircle, partial circle, or a complete or near complete circle) around, e.g., the annulus of a native heart valve (not shown in FIG. 18 for clarity), such that the contraction of the tensioning line 858 reduces the size of the annulus (or other target anatomy/tissue). For annuloplasty implementations, such contraction can improve coaptation of the native heart valve leaflets. It may be necessary or desirable to space the anchors 855 appropriately to allow for better contraction and reshaping of the implant 850 and annulus. In some implementations, spacers 861 may be disposed in a floating manner about the line 858 between anchors 855 to prevent the anchors from being implanted or pulled too closely to one another. However, if the tensioning line 858 were to break during tensioning, the spacers 861 might be released from the line 658 and may embolize, which may necessitate undesirable surgical intervention and/or present severe risks to the patient's health. Therefore, where spacers are implemented, tension monitoring may be particularly important.

Proper anchor spacing and other aspects of implant positioning can be facilitated by integrating echogenic/radiopaque filaments or elements in a tensioning line to allow for better visualization of the implant device/assembly 850. The terms “echogenic” and “radiopaque” are used herein according to their broad and ordinary meanings and may refer to a material that at least partially inhibits, prevents, and/or blocks the passage of electromagnetic radiation therethrough, and therefore is detectable to some degree by an imaging device using an x-ray or other penetrating wave or particle technologies, such as neutron beams or gamma rays, fluoroscopy, MRI, infrared, near-infrared, laser, electromagnetic or radio waves technologies, and the like. The term “radiolucent” is used herein according to its broad and ordinary meaning and may refer to materials that allow/enable the passage of electromagnetic radiation therethrough, and therefore are transparent, or at least partially transparent, to scanning devices using an x-ray or other penetrating wave or particle technologies. Individual filaments of cable constructions disclosed herein can be coated with radiopaque coating, wherein the coating comprises at least one biocompatible metal material, selected from: gold, platinum, titanium, silver, tantalum, barium, bismuth, iridium, tungsten, rhenium, osmium, iridium, palladium, and biocompatible oxides and combinations thereof.

With further reference to FIG. 18, manufacturing the of tensioning line 858 in a cable-type configuration, as shown in FIG. 18, can provide necessary or desirable mechanical stability/strength for the line 858. For example, it may be necessary or desirable for the line 858 to meet certain structural parameters with respect to break load (e.g., 20N minimum when pulled at 90° degrees against a 0.125 mm stainless steel radius), fatigue resistance (e.g., for 3N-7N cyclic loads), outer diameter (e.g., 0.4 mm maximum), as well as parameters relating to visibility under fluoroscopy, elongation resistance at service loads (e.g., in the range of 15-20N), friction, etc.

In some implementations, the cable line 858 is formed of at least one material, selected from metal materials, synthetic polymers, natural fibers, and combinations thereof. In some implementations, the cable 858 is formed of a metal material, selected from titanium, nitinol, platinum, stainless steel, and alloys and combinations thereof.

The cable line 858 can be formed of a plurality of strands/wires 891 that are woven or interlaced circumferentially around a central strand/wire in order to form the wire assembly 858. In some implementations, each of the strands 891 is formed of a plurality of filaments that are twisted/wound around a central filament to form the wound strands 891. Although certain images are shown, it should be understood that the cable 858 can be formed using any suitable winding, twisting, and/or weaving patterns for the groups of filaments and strands.

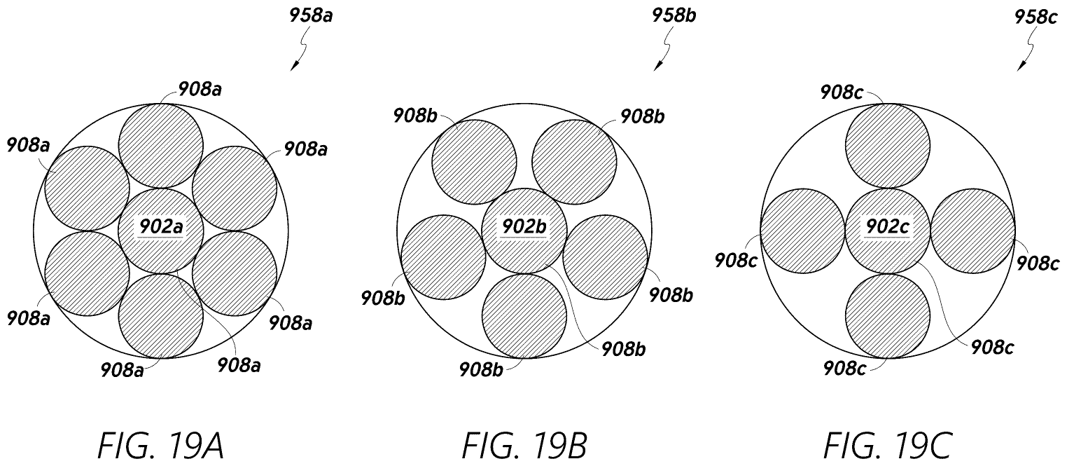

FIGS. 19A-19C show cross-sectional views of tensioning lines/cables 958 that are formed of a plurality of strands/wires 908 combined (e.g., wound, braided) together. In some implementations, each of the strands can be formed of a plurality of filaments combined/wound together. In some implementations, tensioning lines comprise a central strand 902 and at least two additional strands 908 (e.g., four, five, or six additional strands), wherein the at least two additional strands 908 are woven around the central strand 902. In some implementations, the additional strands 908 are woven or interlaced radially in an in-and-out fashion around wire 902 in order to form the line assembly 958. The terms “woven.” “interweave,” and “interlace” are used according to their broad and ordinary meanings, and such terms may be used interchangeably in some contexts to refer to interlacing/intermixing or braiding or winding of a plurality of distinct wires in order to form a single complex pattern. The act of weaving can be performed utilizing various weaving or braiding techniques or patterns known in the art.

In some implementations, each one of the at least two additional strands/wires 908 is identical to the central strand 902. In some implementations, the strand 902 and/or each additional strand 908 is formed filaments of a metal material, selected from titanium, nitinol, platinum, stainless steel, and alloys and combinations thereof. In some implementations, a line assembly 958c comprises a central strand 902c and at least four additional strands 908 woven around the wire 902c, as illustrated at FIG. 19C. In some implementations, at least five additional wires 908b are woven around the central wire 902b, as illustrated at FIG. 19B. In some implementations, at least six additional wires 908a are woven around wire 902a, as illustrated at FIG. 19A.

Advantageously, the plurality of additional wires 908 woven around wire 902 of the line 958 may reduce the risk of grinding or wearing of at least the central wire 902 against the eyelets/couplers of various anchors during implantation and/or contraction procedures, and/or post-operatively as implanted. Therefore, where the central wire 902 comprises one or more variable-resistance, signal, or radiopaque-coated filaments, such filaments can be protected from abrasion and/or damage. For cable-type tensioning lines, one or more filaments of any of the strands/wires that form the structure thereof can be implemented as a variable-resistance strain-indicating filament, a signal (e.g., optical) filament, or a radiopaque filament (e.g., coating or core of filament is radiopaque). In some implementations, a sleeve, such as a polyethylene (PET) sleeve encases the cable 958 and/or the individual strands 908. Such a sleeve can help reduce friction and/or shield the cable. In some implementations, the sleeve can comprise a braided PET cloth.

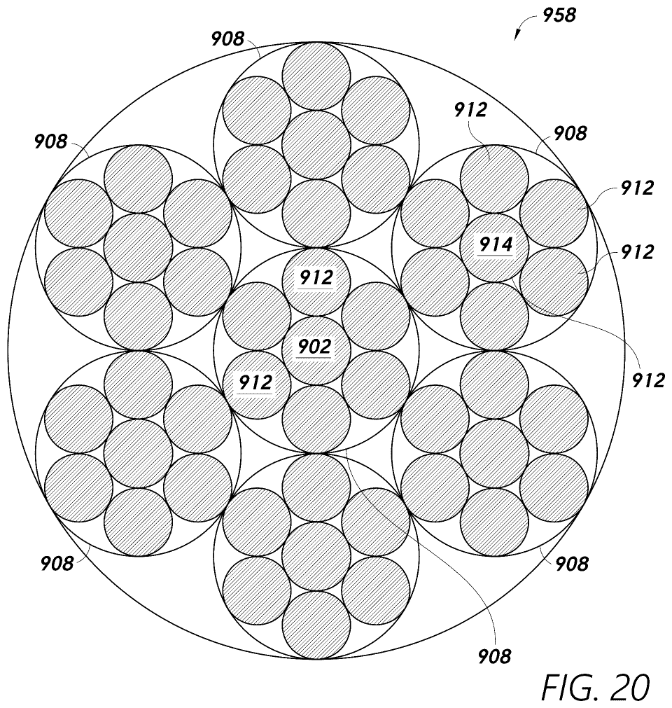

Reference is now made to FIG. 20. In some implementations, each one of the plurality of strands/wires 908 of a cable-type tensioning line 958 can comprise a plurality of filaments 912. For each strand/wire 908, the plurality of inner filaments 912 can be woven around an inner filament 914. The tensioning line 958 shown in FIG. 20 can comprise a central strand/wire 902, formed of a plurality of wound filaments 912, and at least four (e.g., five or six) outer strands 908, each formed of a plurality of wound filaments 912, as illustrated. The filaments 912 that form the various strands 908 of the cable 958 can comprise a metal material, such as titanium, nitinol, stainless steel, and combinations thereof.

In some implementations, the filaments 912 comprise cobalt chrome filaments having a platinum core for radiopacity. In some implementations, the diameter of each of the inner filaments 912 is in the range of about 0.001 to about 0.1 mm, such as between 0.01-0.08 mm and/or between 0.02-0.04 mm. In some implementations, the diameter is about 0.03 mm.

As described above, any of the filaments 912 of the cable 958 can comprise a variable-resistance strain gauge conductor, a signal filament (e.g., fiberoptic filament), and/or a radiopaque core and/or coating. For ease of description, any such types of filaments are referred to below as a ‘special’ filament; it should be understood that any reference below to a ‘special’ filament may be understood to represent a radiopaque filament (e.g., coated with a radiopaque coating or having a radiopaque core or body), a variable-resistance strain gauge filament, or any other type of signal-carrying filament, such as a fiberoptic filament, metal conductor, or the like.

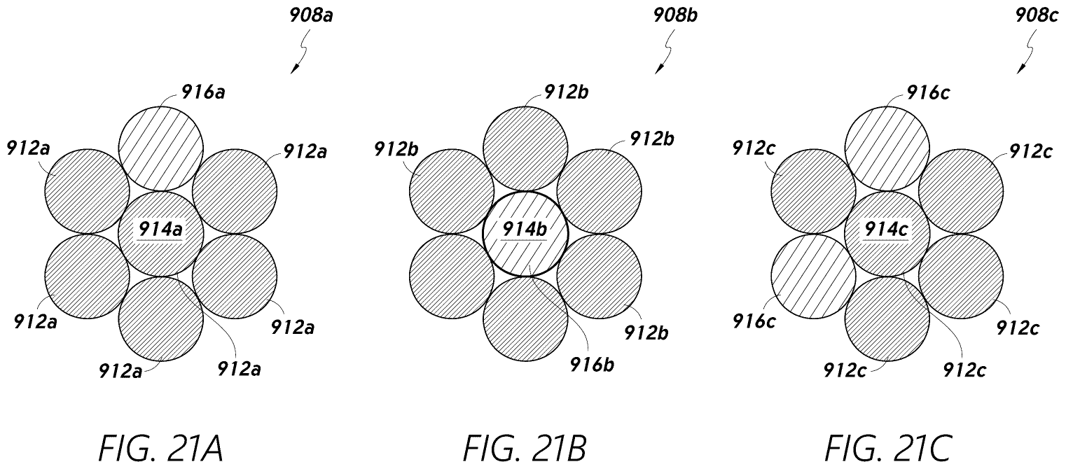

FIGS. 21A-21C show groupings of filaments 912, which can be collectively wound or braided together to form a strand/wire of a tensioning line in accordance with one or more implementations of the present disclosure. In the example of FIG. 21A, a single outer filament 916a of the strand 908a is implemented as a special filament, whereas the remaining filaments 912a, including the central filament 914a, are standard (e.g., stainless steel, cobalt/chrome, or the like) filaments. In the example of FIG. 21B, a single central filament 916b of the strand 908b is implemented as a special filament, whereas the remaining outer filaments 912b are standard filaments.

In the example of FIG. 21C, multiple filaments 916c of the group of filaments 912c are implemented as special filaments. Although both special filaments 916c shown are outer filaments, it should be understood that one of the special filaments can be the central filament 914c. Furthermore, although two special filaments 916c are shown, it should be understood that any number of the filaments can be implemented as special filaments. In some implementations, all of the filaments 912 are special filaments. In implementations comprising strands with multiple special filaments, different ones of the special filaments can be different types of special filaments. That is, the various types of special filaments described herein can be combined in a single strand, or in separate strands of a single cable.

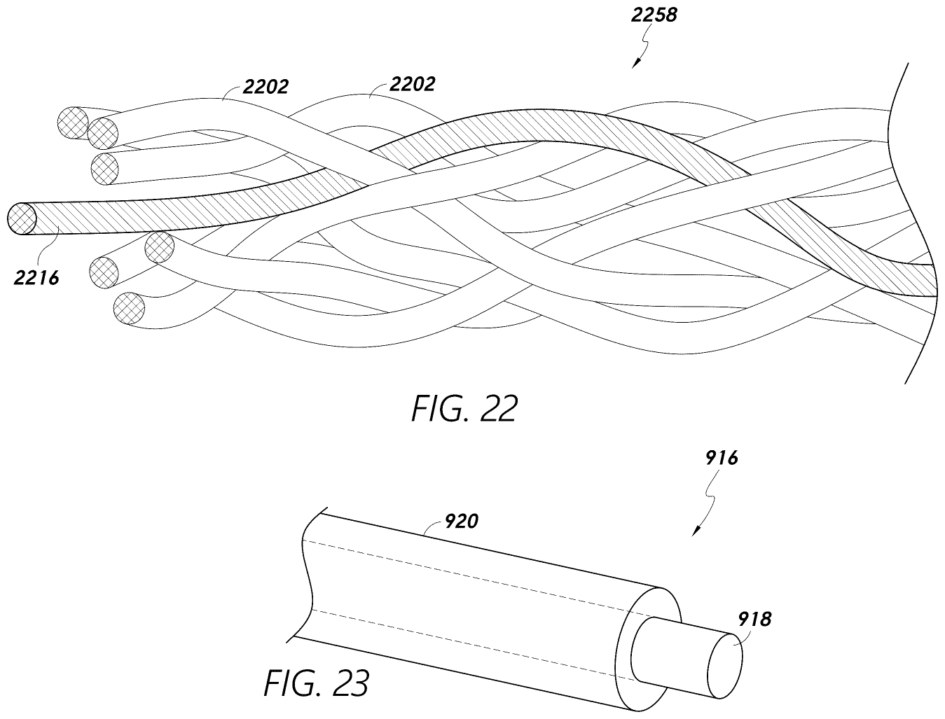

Reference is now made to FIG. 22. In some implementations, there is provided a tensioning line 2258 comprising a plurality of main filaments 2202 and at least one special filament 2216, wherein the plurality of main filaments 2202 and the at least one special filament 2216 are woven/braided or interlaced together. In some implementations, the main filaments 2202 and the special filament(s) 2216 are randomly woven or interlaced around each other, thereby forming a random interlaced structure. Any of the implementations of tensioning lines including one/or more special filaments or strands can include filaments or strands woven/braided as shown in FIG. 22 or in any other suitable or desirable interlacing configuration.

Reference is not made to FIG. 23. The various types of filaments disclosed herein can be implemented as comprising an inner core 918 comprising at least one first material, and an outer sheath/tube 920 comprising at least one second material, wherein said inner core 918 is disposed within a lumen of the outer sheath 920. In some implementations, the outer sheath 920 is configured to provide strength, durability, and biocompatibility to the composite tube 916, while the inner core 918 is configured to provide enhanced resiliency, electrical conduction, and/or radiopaque functionality. In some implementations, the first material is selected from: gold, platinum, titanium, silver, tantalum, palladium, cobalt, cobalt chrome, nitinol, and combinations and alloys thereof. In some implementations, the first material is radiopaque. In some implementations, the first material comprise gold. In some implementations, the second material is selected from: titanium, stainless steel, gold, platinum, nitinol, and combinations and alloys thereof. In some implementations, the second material comprises at least one metal alloy.

The tensioning wires, lines, contracting members, and other lines described herein can be implemented as and/or include the features of any of assemblies of FIGS. 19A-19C, 20, 21A-21C, 22, and/or 23. Furthermore, it is to be understood that any of the tensioning lines and/or associated components/features of FIGS. 19A-19C. 20, 21A-21C. 22, and/or 23 can be utilized in combination with any of the implants described hereinabove.

### Sterilization

Any of the various systems, assemblies, devices, components, apparatuses, etc. in this disclosure (including in the examples below) can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

### Additional Description of Examples

Provided below is a list of examples, each of which may include aspects of any of the other examples disclosed herein. Furthermore, aspects of any example described above may be implemented in any of the numbered examples provided below.

Example 1: A tensioning device comprising a handle, a line carriage configured to be coupled to a tensioning line, and a manually-manipulatable actuator configured to be selectively transitioned between a fine-tensioning configuration and a gross-tensioning configuration.

Example 2: The tensioning device of any example disclosed herein, in particular example 1, wherein the actuator comprises an internal channel configured to have a bolt member associated with the line carriage disposed therein, the internal channel including threading configured to mesh with threading associated with the bolt member.

Example 3: The tensioning device of any example disclosed herein, in particular example 2, wherein in the fine-tensioning configuration, threading of the bolt is meshed with the threading associated with the actuator, and in the gross-tensioning configuration, the threading of the bolt is not meshed with the threading associated with the actuator.

Example 4: The tensioning device of any example disclosed herein, in particular example 3, wherein the actuator is configured to transition between the fine-tensioning configuration and the gross-tensioning configuration by tilting relative to an axis of the tensioning device.

Example 5: The tensioning device of any example disclosed herein, in particular example 4, wherein the internal channel comprises two merged pseudo channels that are angled relative to one another.

Example 6: The tensioning device of any example disclosed herein, in particular example 5, wherein the two merged pseudo channels include a first pseudo channel that is parallel to an axis of the actuator and a second pseudo channel that is angled relative to the axis of the actuator.

Example 7: The tensioning device of any example disclosed herein, in particular example 5, wherein the threading of the actuator is associated with only a first pseudo channel of the two merged pseudo channels.

Example 8: The tensioning device of any example disclosed herein, in particular example 7, wherein in a first axial portion of the first pseudo channel, the threading is associated only with a first diametrical half of the first pseudo channel, and in a second axial portion of the first pseudo channel, the threading is associated only with a second diametrical half of the first pseudo channel that is opposite the first diametrical half.

Example 9: The tensioning device of any example disclosed herein, in particular any of examples 3-8, wherein the actuator comprises a first axial segment configured to rotate about an axis of the actuator relative to a second axial segment of the actuator.

Example 10: The tensioning device of any example disclosed herein, in particular example 9, wherein each of the first axial segment and the second axial segment comprise internal threading associated with only one diametrical half thereof.

Example 11: The tensioning device of any example disclosed herein, in particular example 10, wherein in the fine-tensioning configuration, the internal threading of the first axial segment does not circumferentially overlap with the internal threading of the second axial segment, and in the gross-tensioning configuration, the internal threading of the first axial segment circumferentially overlaps with the internal threading of the second axial segment.

Example 12: The tensioning device of any example disclosed herein, in particular example 11, wherein in the gross-tensioning configuration, at least one of the bolt member or the actuator is biased such that the bolt member contacts non-threaded internal walls of the first and second axial segments, such that the bolt member can axially slide within the actuator.

Example 13: The tensioning device of any example disclosed herein, in particular any of examples 1-12, wherein the tensioning line comprises one or more filaments having variable electrical resistance.

Example 14: The tensioning device of any example disclosed herein, in particular example 13, wherein an electrical resistance of the one or more filaments changes based on an amount of stretch in the tensioning line.

Example 15: A tensioning device comprising a handle, a line carriage configured to be coupled to a tensioning line, and an electrical motor assembly configured to axially translate the tensioning line relative to the handle to thereby modify a tension in the tensioning line.

Example 16: The tensioning device of any example disclosed herein, in particular example 15, wherein the electrical motor assembly comprises a motor gear meshed with a gear rotationally fixed to an internally-threaded rotational-to-linear translation structure.

Example 17: The tensioning device of any example disclosed herein, in particular example 16, wherein the electrical motor assembly further comprises an externally-threaded axial translating rod, the rod being coupled to the line carriage.

Example 18: The tensioning device of any example disclosed herein, in particular example 17, wherein the tensioning line is configured to be disposed through an inner channel of the translating rod and the line carriage.

Example 19: The tensioning device of any example disclosed herein, in particular any of examples 15-18, further comprising an electrical tension-measuring means configured to generate signals indicating the tension in the tensioning line.

Example 20: The tensioning device of any example disclosed herein, in particular example 19, further comprising a user output means configured to provide user output indicating a tension condition of the tensioning line.

Example 21: The tensioning device of any example disclosed herein, in particular example 19, wherein the tension-measuring means comprises one or more contacts configured to provide signals indicating an electrical resistance of the electrical motor assembly.

Example 22: The tensioning device of any example disclosed herein, in particular example 19, wherein the tension-measuring means comprises a strain gauge.

Example 23: The tensioning device of any example disclosed herein, in particular example 22, wherein the strain gauge is physically coupled between the tensioning line and the line carriage.

Example 24: The tensioning device of any example disclosed herein, in particular any of examples 15-23, wherein the tensioning line comprises one or more filaments having an electrical resistance that varies based on an amount of stretch in the tensioning line.

Example 25: The tensioning device of any example disclosed herein, in particular example 24, wherein the one or more filaments are braided with sutures or wires.

Example 26: The tensioning device of any example disclosed herein, in particular any of examples 15-25, wherein the tensioning line further comprises one or more signal filaments.

Example 27: The tensioning device of any example disclosed herein, in particular example 26, wherein the one or more signal filaments are fiber optic filaments.

### Additional Implementations

Depending on the implementation, certain acts, events, or functions of any of the processes or algorithms described herein can be performed in a different sequence, can be added, merged, or left out altogether. Thus, in some implementation, not all described acts or events are necessary for the practice of the processes.

Further, the techniques, methods, operations, steps, etc. described or suggested herein or in the references incorporated herein can be performed on a living subject (e.g., human, other animal, etc.) or on a simulation, such as a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without author input or prompting, whether these features, elements and/or steps are included or are to be performed in any particular implementation. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain implementations require at least one of X, at least one of Y and at least one of Z to each be present.

It should be appreciated that in the above description of implementations, various features are sometimes grouped together in a single example. Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular implementation herein can be applied to or used with any other implementation(s). Further, no component, feature, step, or group of components, features, or steps are necessary or indispensable for each implementation. Thus, it is intended that the scope of the inventions herein disclosed and claimed below should not be limited by the particular implementations described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example implementations belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A tensioning device comprising:
a handle;
a line carriage configured to be coupled to a tensioning line; and
an electrical motor assembly configured to axially translate the tensioning line relative to the handle to thereby modify a tension in the tensioning line.

2. The tensioning device of claim 1, wherein the electrical motor assembly comprises a motor gear meshed with a gear rotationally fixed to an internally-threaded rotational-to-linear translation structure.

3. The tensioning device of claim 2, wherein the electrical motor assembly further comprises an externally-threaded axial translating rod, the axial translating rod being coupled to the line carriage.

4. The tensioning device of claim 3, wherein the tensioning line is configured to be disposed through an inner channel of the axial translating rod and the line carriage.

5. The tensioning device of claim 1, further comprising an electrical tension-measuring means configured to generate signals indicating the tension in the tensioning line.

6. The tensioning device of claim 5, further comprising a user output means configured to provide user output indicating a tension condition of the tensioning line.

7. The tensioning device of claim 5, wherein the electrical tension-measuring means comprises one or more contacts configured to provide signals indicating an electrical resistance of the electrical motor assembly.

8. The tensioning device of claim 5, wherein the electrical tension-measuring means comprises a strain gauge.

9. The tensioning device of claim 8, wherein the strain gauge is physically coupled between the tensioning line and the line carriage.

10. The tensioning device of claim 1, wherein the tensioning line comprises one or more filaments having an electrical resistance that varies based on an amount of stretch in the tensioning line.

11. The tensioning device of claim 10, wherein the one or more filaments are braided with sutures or wires.

12. The tensioning device of claim 1, wherein the tensioning line further comprises one or more signal filaments.

13. The tensioning device of claim 12, wherein the one or more signal filaments are fiber optic filaments.

14. A tensioning device comprising:
a handle;
a line carriage configured to be coupled to a tensioning line; and
a manually-manipulatable actuator configured to be selectively transitioned between a fine-tensioning configuration and a gross-tensioning configuration.

15. The tensioning device of claim 14, wherein the actuator comprises an internal channel configured to have a bolt member associated with the line carriage disposed therein, the internal channel including threading configured to mesh with threading associated with the bolt member.

16. The tensioning device of claim 15, wherein:
in the fine-tensioning configuration, threading of the bolt member is meshed with the threading associated with the actuator; and
in the gross-tensioning configuration, the threading of the bolt member is not meshed with the threading associated with the actuator.

17. The tensioning device of claim 16, wherein the actuator is configured to transition between the fine-tensioning configuration and the gross-tensioning configuration by tilting relative to an axis of the tensioning device.

18. The tensioning device of claim 17, wherein the internal channel comprises two merged pseudo channels that are angled relative to one another.

19. The tensioning device of claim 18, wherein the two merged pseudo channels include a first pseudo channel that is parallel to an axis of the actuator and a second pseudo channel that is angled relative to the axis of the actuator.

20. The tensioning device of claim 18, wherein the threading of the actuator is associated with only a first pseudo channel of the two merged pseudo channels.
